(19) United States
(12) Patent Application Publication
(10) Pub. No.: US 2007/0178051 A1
(43) Pub. Date: Aug. 2, 2007

(54) STERILIZED NANOPARTICULATE GLUCOCORTICOSTEROID FORMULATIONS

(75) Inventors: John Pruitt, Collegeville, PA (US); Raj Kewalramani, Collegeville, PA (US); David Slifer, Royersford, PA (US); Jack Michael Shaw, Conowingo, MD (US); Stephen B. Ruddy, Schwenksville, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International, Ltd.

(21) Appl. No.: 11/275,775

(22) Filed: Jan. 27, 2006

(51) Int. Cl. A61K 31/573; A61K 9/14; A61K 9/48; A61K 9/20; A61K 9/22

(52) U.S. Cl. 424/46; 424/451; 424/464; 424/468; 514/179

(57) ABSTRACT

The invention is directed sterile to compositions of glucocorticosteroids useful in the prophylaxis and chronic treatment of asthma and other allergic and inflammatory conditions in adults and pediatric patients.

### FIELD OF THE INVENTION

The invention is directed generally to sterile compositions useful in the prophylaxis and chronic treatment of asthma in adults and pediatric patients and for the relief of symptoms of allergic conjunctivitis and seasonal allergic rhinitis in adults and pediatric patients. The sterile compositions comprise a glucocorticosteroid. The invention is also directed to pharmaceutical compositions of the same useful for parenteral, inhalation, and topical administration for the treatment of a variety of inflammatory and allergic conditions.

### BACKGROUND OF THE INVENTION

### A. Background Regarding Glucocorticosteroids

Glucocorticosteroids have been shown to be effective for the maintenance treatment of asthma as a prophylactic therapy, for the management of the nasal symptoms of seasonal and perennial allergic and nonallergic rhinitis in adults and pediatric patients, and for the relief of the signs and symptoms of seasonal allergic conjunctivitis.

U.S. Pat. No. 6,392,036 to Karlsson et al., for “Dry Heat Sterilization of Glucocorticosteroid,” refers to a process for the sterilization of a dry powder comprising a glucocorticosteroid. The process comprises dry heat treating the powder at a temperature of from 100 to 130 degrees centigrade. This process is disclosed for the sterilization of budesonide powder followed by aseptic addition of liquids and excipients to prepare the product, Pulmicort Respules. The patent also teaches that sterilization in the presence of water (i.e. moist heat sterilization) is not an acceptable method for sterilization because of particle agglomeration. Further, ethylene oxide is not an acceptable process for sterilization because of the generation of toxic residues. Moreover, beta and gamma irradiation as a process for sterilization of micronized budesonide demonstrated significant chemical breakdown at low radiation exposure levels.

U.S. Pat. No. 6,464,958 to Bernini et al., for “Process for the Preparation of Suspensions of Drug Particles for Inhalation Delivery,” refers to a process for making therapeutically acceptable sterile micronized beclomethasone dipropionate as a result of gamma irradiation. The reference discloses that beclomethasone dipropionate, when subjected to gamma-irradiation at 2 to 9 KGy under particular conditions, remains chemically stable. The irradiation is carried out in a polythene container having replaced air with nitrogen and sealed in two oxygen-proof materials, Polikem bags. The sterilized micronized beclomethasone dipropionate is processed in aseptic fashion using a turbo-emulsifier in which the aqueous contents and excipients were previously sterilized via steam sterilization using a steam jacket.

European Patent Application No. EP 1 454 636 A1 to Gentile et al., for “Sterilization of Glucocorticoid Drug Particles for Pulmonary Delivery,” refers to a process for the steam sterilization of glucocorticosteroids comprising heating a mixture of micronized glucocorticosteroids and water at a temperature ranging between 100 and 130 degrees centigrade. The glucocorticosteroid/water ratio is selected in a range between 3:100 to 10:100. Preferred glucocorticosteroids are beclomethasone or beclomethasone dipropionate. Preferred sterilization is at 121° C. for 20 min. The impurity profile of the sterilized glucocorticosteroid suspensions of the invention are not significantly different from the profile of the non-sterilized glucocorticosteroid.

U.S. Pat. No. 6,039,932 to Govind et al., for “Medicinal Inhalation Aerosol Formulations Containing Budesonide,” describes a propellant-based glucocorticosteroid formulation. Claimed preferred surfactants include oleic acid, sorbitan oleates, and lecithin.

International Patent Application WO 98/00111 to Waldrep et. al., for “High Dose Liposomal Aerosol Formulations,” refers to a high dose budesonide-liposome aerosol composition comprising up to about 12.5 mg/ml budesonide in up to about 187.5 mg of dilaurolyphosphatidylcholine/ml. Other phospholipids useful in the practice of the described process can be selected from a group consisting of egg yolk phosphatidyl-choline, hydrogenated soybean phosphatidylcholine, dilauroylphosphatidylcholine, dimyristoylphosphatidylcholine, dioleoylphosphatidylcholine, and dipalmitoyl phosphatidylcholine.

U.S. Pat. No. 5,091,188 by Haynes, for” Phospholipid-coated microcrystals: injectable formulations of water-insoluble drugs,” refers to the preparation of a syringable, injectable pharmaceutical composition consisting of a suspension of solid particles of a water-insoluble pharmacologically active substance on the order of about 50 nm to about 10,000 nm, coated with a layer of membrane-forming amphipathic lipid (phospholipid). The composition is also described for inhalation and administration in the eye. The drug substance is reduced in particle size via a process involving sonication or high shear in the presence of the phospholipid.

U.S. Pat. No. 6,863,865 by McAffer et al., for “Sterilization of pharmaceuticals,” discloses the successful sterilization of a glucocorticosteroid (budesonide) formulation using a rapid elevation to high temperature with hold followed by rapid return to ambient temperature (also described at High Temperature Short Time Sterilization, “HTST Sterilization”). The HTST sterilization cycle did not result in an increase in the levels of impurities in the budesonide formulation and the physical properties of the formulation were not altered.

U.S. Pat. No. 6,139,870 by Verrecchia, for “Stabilized nanoparticles which are filterable under sterile conditions,” discloses a process for the sterile filtration of a nanoparticle suspension comprising one hydrophobic, water-insoluble and water indispersible polymer or copolymer emulsified in an aqueous phase comprising a phospholipid and an oleic acid salt. The nanoparticles contain a pharmaceutical agent, with focus on the “taxoid family” and an injectable composition.

U.S. Pat. No. 5,922,355 by Parikh et al., for “Composition and method of preparing microparticles of water-insoluble substances,” discloses a probe sonicator technique in which poorly water-insoluble drugs are prepared in submicron particle size when combined with one or more surface modifiers or surfactants together with natural or synthetic phospholipids. The combination surface modifier or surfactant and a phospholipid approach generates a final particle size at least one-half smaller as compared to that obtained when using phospholipid alone. The phospholipids may be phosphatidylcholine, phosphatidylethanolamine, phosphatidylserine, phosphatidylinositol, phosphatidyltglycerol, phosphatidic acid, lysophopholipids, egg or soybean phonpholipid (natural, partially or fully hydrogenated).

U.S. Pat. No. 5,858,410 by Muller et al., for “Pharmaceutical nanosuspensions for medicament administration as systems with increased saturation solubility and rate of solution,” discloses the preparation of drug carrier particles containing at least one sparingly soluble therapeutic compound in the particle size range of 10 to 1000 nm. Natural occurring surfactants include phospholipids (lecithins, phospholipids, sphingolipids, sterols, egg lecithin, soya lecithin, and hydrogenated lecithins are utilized to stabilize the system along with other dispersion-stabilizing substances (e.g. poloxamers, mono & diglycerides, poloxamines, sugar alcohols, alkylphenols)). Medicaments described in the patent include corticoids (e.g., aldosterone, triamcinolone, and dexamethasone). The device utilized by Muller in producing the small particles was a Microfluidizer or Nanojet, a process for creating high shear of liquids in a jet stream.

U.S. Pat. No. 5,993,781 by Snell et. al., for “Fluticasone Propionate Nebulizable Formulations,” refers to bulk suspensions of fluticasone propionate sterilized via steam.

European Patent Application No. EP 1 310 243 A1 to Santesson et. al., for “Novel Formulation,” refers to a metered unit dose comprising 32 μg of budesonide, wherein the budesonide is produced as fine particles which are suspended in an aqueous medium with a pH in the range of 3.5 to 5.0. Preferably, the formulation contains the chelating agent EDTA at about 0.005 to 0.1% w/w.

U.S. Pat. No. 5,914,122 to Otterbeck et al., for “Stable Budesonide Solutions, Method of Preparing Them and Use of These Solutions As Enema Preparations And Pharmaceutical Foams,” notes that the stability of budesonide solutions critically depends on the pH (claim pH <6). Budesonide stability is enhanced in the presence of EDTA or cyclodextrins.

U.S. Published Patent Application No. 2002/0037257 A1 to Fraser et al., for “Budesonide Particles and Pharmaceutical Compositions Containing Them,” stresses the importance of crystalline budesonide particles having a “smooth surface” with BET values from 1 to 4.5 m²/g. The described process uses a super-critical fluid.

### B. Background Regarding Nanoparticulate Compositions

Nanoparticulate compositions, first described in U.S. Pat. No. 5,145,684 (“the '684 patent”), are particles consisting of a poorly soluble therapeutic or diagnostic agent having adsorbed onto, or associated with, the surface thereof a non-crosslinked surface stabilizer.

Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;”U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” U.S. Pat. No. 6,592,903 for “Nanoparticulate Dispersions Comprising a Synergistic Combination of a Polymeric Surface Stabilizer and Dioctyl Sodium Sulfosuccinate,” U.S. Pat. No. 6,582,285 for “Apparatus for sanitary wet milling;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof,” U.S. Pat. No. 6,811,767 for “Liquid droplet aerosols of nanoparticulate drugs;” and U.S. Pat. No. 6,908,626 for “Compositions having a combination of immediate release and controlled release characteristics;” all of which are specifically incorporated by reference. In addition, U.S. Patent Application No. 20020012675 A-1, published on Jan. 31, 2002, for “Controlled Release Nanoparticulate Compositions,” and WO 02/098565 for “System and Method for Milling Materials,” describe nanoparticulate compositions, and are specifically incorporated by reference.

Amorphous small particle compositions are described, for example, in U.S. Pat. Nos. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Nanoparticulate glucocorticosteroids are described, for example, in U.S. Pat. No. 6,264,922 for “Aerosols Containing Nanoparticulate Dispersions,” U.S. Pat. No. 5,747,001 for “Aerosols Containing Beclomethasone Nanoparticle Dispersions;” U.S. 20040208833 A1 to Hovey et al., for “Novel fluticasone formulations,” US 20040057905 A1 to Wood et al., for “Nanoparticulate beclomethasone dipropionate compositions,” US 20040141925 to Bosch et al., for “Novel triamcinolone compositions,” and US 20030129242 to Bosch et al., for “Sterile filtered nanoparticulate formulations of budesonide and beclomethasone having tyloxapol as a surface stabilizer.”

### C. Background Relating to Sterilization of Nanoparticulate Active Agent Compositions

There are several commonly used methods for sterilizing pharmaceutical products: heat sterilization, sterile filtration, and ethylene oxide exposure.

1. Heat Sterilization of Nanoparticulate Active Agent Compositions

One of the problems that may be encountered with heat sterilization of nanoparticulate active agent compositions is the solubilization and subsequent recrystallization of the component active agent particles. This process results in an increase in the size distribution of the active agent particles. In cases where the nanoparticulate active agent formulations contain surface stabilizers, which have cloud points lower than the sterilization temperature (generally about 121° C.), the surface stabilizers may desorb or disassociate from the nanoparticulate active agent surfaces and precipitate from solution at or below the sterilization temperature. Thus, some nanoparticulate active agent formulations also exhibit particle aggregation following exposure to elevated temperatures during the heat sterilization process.

Crystal growth and particle aggregation in nanoparticulate active agent preparations are highly undesirable for several reasons. The presence of large crystals in the nanoparticulate active agent composition may cause undesirable side effects, especially when the preparation is in an injectable formulation. This is also true for particle aggregation, as injectable formulations preferably have an effective average particle size of greater than about 250 nm. Larger particles formed by particle aggregation and recrystallization, such as particles having a size of greater than 2 microns, can interfere with blood flow, causing pulmonary embolism and death.

In addition, with both injectable and oral formulations the presence of large crystals, and therefore varying particle sizes, and/or particle aggregation can change the pharmacokinetic profile of the administered active agent. For oral formulations, the presence of large crystals or aggregates creates a variable bioavailability profile because smaller particles dissolve faster than the larger aggregates or larger crystal particles. A faster rate of dissolution is associated with greater bioavailability and a slower rate of dissolution is associated with a lower bioavailability. This is because bioavailability is proportional to the surface area of an administered drug and, therefore, bioavailability increases with a reduction in the particle size of the dispersed agent (see U.S. Pat. No. 5,662,833).

With a composition having widely varying particle sizes, bioavailability becomes highly variable and inconsistent and dosage determinations become difficult. Moreover, because such crystal growth and particle aggregation are uncontrollable and unpredictable, the quality of the nanoparticulate compositions is inconsistent. For intravenously injected particulate formulations, the presence of large crystals or aggregates can induce an immune system response which causes the larger particles to be transported by macrophage cells to the liver or spleen and metabolized, in addition to the embolytic effects described above.

For inhaled particulate compositions, particle size is also critical as the particle size determines the delivery site. Pulmonary drug delivery is accomplished by inhalation of an aerosol through the mouth and throat. Particles having aerodynamic diameters of greater than about 5 microns generally do not reach the lung; instead, they tend to impact the back of the throat and are swallowed and possibly orally absorbed. Particles having diameters of about 2 to about 5 microns are small enough to reach the upper- to mid-pulmonary region (conducting airways), but are too large to reach the alveoli. Even smaller particles, i.e., about 0.5 to about 2 microns, are capable of reaching the alveolar region. Particles having diameters smaller than about 0.5 microns can also be deposited in the alveolar region by sedimentation, although very small particles may be exhaled.

As taught by U.S. 20020102294 A1, conventional techniques are extremely inefficient in delivering agents to the lung for a variety of reasons. For example, it has been reported that ultrasonic nebulization of a suspension containing fluorescein and latex drug spheres, representing insoluble drug particles, resulted in only 1% aerosolization of the particles, while air-jet nebulization resulted in only a fraction of particles being aerosolized. Susan L. Tiano, “Functionality Testing Used to Rationally Assess Performance of a Model Respiratory Solution or Suspension in a Nebulizer,” Dissertation Abstracts International, 56/12-B, pp. 6578 (1995). Another problem encountered with nebulization of liquid formulations was the long (4-20 min) period of time required for administration of a therapeutic dose. Long administration times are required because conventional or non-nanoparticulate liquid formulations for nebulization are very dilute solutions or suspensions of micronized drug substance. Prolonged administration times are undesirable because they lessen patient compliance and make it difficult to control the dose administered. Lastly, aerosol formulations of micronized drug are not feasible for deep lung delivery of water-insoluble compounds because the droplets needed to reach the alveolar region (0.5 to 2 microns) are too small to accommodate micronized drug crystals, which are typically 2-3 microns or more in diameter.

Conventional pressurized metered dose inhalers (pMDIs) are also inefficient in delivering drug substance to the lung. In most cases, pMDIs consist of suspensions of micronized drug substance in halogenated hydrocarbons such as chlorofluorocarbons (CFCs) or hydrofluoroalkanes (HFAs). Actuation of the pMDI results in delivery of a metered dose of drug and propellant, both of which exit the device at high velocities because of the propellant pressures. The high velocity and momentum of the drug particles results in a high degree of oropharyngeal impaction as well as loss to the device used to deliver the agent. These losses lead to variability in therapeutic agent levels and poor therapeutic control. In addition, oropharyngeal deposition of drugs intended for topical administration to the conducting airways (such as corticosteroids) can lead to systemic absorption with resultant undesirable side effects. Additionally, conventional micronization (air-jet milling) of pure drug substance can reduce the drug particle size to no less than about 2-3 microns. Thus, the micronized material typically used in pMDIs is inherently unsuitable for delivery to the alveolar region and is not expected to deposit below the central bronchiole region of the lung.

Delivery of dry powders to the lung utilizing micronized drug substance is also problematic. In the dry powder form, micronized substances tend to have substantial interparticle electrostatic attractive forces which prevent the powders from flowing smoothly and generally make them difficult to disperse. Thus, two key challenges to pulmonary delivery of dry powders are the ability of the device to accurately meter the intended dose and the ability of the device to fully disperse the micronized particles. For many devices and formulations, the extent of dispersion is dependent upon the patient's inspiration rate, which itself may be variable and can lead to a variability in the delivered dose.

Delivery of drugs to the nasal mucosa can also be accomplished with aqueous, propellant-based, or dry powder formulations. However, absorption of poorly soluble drugs can be problematic because of mucociliary clearance which transports deposited particles from the nasal mucosa to the throat where they are swallowed. Complete clearance generally occurs within about 15-20 minutes. Thus, poorly soluble drugs which do not dissolve within this time frame are unavailable for either local or systemic activity.

Aggregation of nanoparticle active agent compositions upon heating is directly related to the precipitation of the surface stabilizer at temperatures above the cloud point of the surface stabilizer. At this point, the bound surface stabilizer molecules are likely to dissociate from the nanoparticles and precipitate, leaving the nanoparticles unprotected. The unprotected nanoparticles then aggregate into clusters of particles.

Several methods have been suggested in the prior art for preventing such crystal growth and particle aggregation following heat sterilization, including adding a cloud point modifier or crystal growth modifier to the nanoparticulate active agent composition and purifying the surface stabilizer. For example, U.S. Pat. No. 5,298,262 describes the use of an anionic or cationic cloud point modifier in nanoparticulate active agent compositions and U.S. Pat. No. 5,346,702 describes nanoparticulate active agent compositions having a nonionic surface stabilizer and a non-ionic cloud point modifier. The cloud point modifier enables heat sterilization of the nanoparticulate active agent compositions with low resultant particle aggregation. U.S. Pat. No. 5,470,583 describes nanoparticulate active agent compositions having a non-ionic surface stabilizer and a charged phospholipid as a cloud point modifier.

The prior art also describes methods of limiting crystal growth in a nanoparticulate active agent composition by adding a crystal growth modifier (see U.S. Pat. Nos. 5,662,883 and 5,665,331). In addition, U.S. Pat. No. 5,302,401 describes nanoparticulate active agent compositions having polyvinylpyrrolidone (PVP) as a surface stabilizer and sucrose as a cryoprotectant (allowing the nanoparticles to be lyophilized). The compositions exhibit minimal particle aggregation following lyophilization.

Another method of limiting particle aggregation or crystal growth of nanoparticulate active agent compositions during sterilization known prior to the present invention was the use of purified surface stabilizers. U.S. Pat. No. 5,352,459 describes nanoparticulate active agent compositions having a purified surface stabilizer (having less than 15% impurities) and a cloud point modifier. Purification of surface stabilizers can be expensive and time consuming, thus significantly raising production costs of compositions requiring such stabilizers to produce a stable nanoparticulate active agent composition.

2. Sterile Filtration

Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize conventional suspensions of micron-sized drug particles because the drug substance particles are too large to pass through the membrane pores. In principle, 0.2 μm filtration can be used to sterilize nanoparticulate active agent compositions. However, because nanoparticulate active agent compositions have a size range, many of the particles of a typical nanoparticulate active agent composition having an average particle size of 200 nm may have a size greater than 200 nm. Such larger particles tend to clog the sterile filter. Thus, only nanoparticulate active agent compositions having very small average particle sizes can be sterile filtered.

3. Ethylene Oxide Method

The ethylene oxide method has been a widely used sterilization method for suspension/dispersion products where product or components are thermolabile. Most of the currently marketed products utilize this technique by which individual components are sterilized using this method and then processed or assembled together aseptically. The technique, however, requires the elimination of residual ethylene oxide from the product, which is a time consuming and difficult process with the possibility of residual ethylene oxide contaminating the final drug product.

4. Gamma Irradiation

US 2004105778 A1 to Lee et al., for “Gamma Irradiation of Solid Dose Nanoparticulate Active Agents,” relates to methods for terminal sterilization of solid forms of nanoparticulate active agent compositions via gamma irradiation. The nanoparticulate active agent has an effective average particle size of less than about 2 microns, prior to incorporation into a solid form for sterilization. The resultant sterilized compositions exhibit excellent redispersibility, homogeneity, and uniformity. Also encompassed are compositions made via the described method and methods of treating animals and humans using such compositions.

WO 2004/105809 to Bosch et al., for Sterilization of Dispersions of Nanoparticulate Active Agents with Gamma Radiation,” relates to methods for sterilization of dispersions of one or more nanoparticulate active agents via gamma irradiation and to the obtainable pharmaceutical compositions.

There remains a need in the art for sterile, stable glucocorticosteroid compositions exhibiting increased pharmaceutical effectiveness. The present invention satisfies this need.

### SUMMARY OF THE INVENTION

The present invention is directed to the unexpected discovery that glucocorticosteroids, in the presence of one or more nonionic surface stabilizers, can be readily heat sterilized without incurring substantial changes in particle size or chemical purity, provided that an amphiphilic lipid is added to the composition prior to the sterilization process step.

The present invention is directed to drug compositions comprising a heat sterilized glucocorticosteroid dispersion or suspension. Such drug compositions are known to be effective for the maintenance treatment of asthma as a prophylactic therapy for the management of the nasal symptoms of seasonal and perennial allergic and non-allergic rhinitis in adults and pediatric patients, and for the relief of the signs and symptoms of seasonal allergic conjunctivitis. The dispersion is formulated as a sterile, pharmaceutical composition of glucocorticosteroid particles suspended in an aqueous vehicle comprising at least one nonionic surface stabilizer and at least one amphiphilic lipid. The glucocorticosteroid particles have an effective average particle size of less than about 2000 nm.

The compositions of the invention comprise aqueous suspensions of glucocorticosteroids (e.g., budesonide, fluticasone propionate, and beclomethasone dipropionate) and at least one nonionic surface stabilizer (e.g., polysorbate 80, tyloxapol, or Lutrol F127 NF) and an amphiphilic lipid (e.g., soy or egg lecithin phosphatides which in addition to the primary constituent phosphatidylcholine must also contain negatively charged phosphatides, such as phosphatidylinositol, phosphatidylserine, phosphatidic acid, phosphatidylglycerol, and the corresponding lysophosphatides). Preferred amphiphilic lipids are those phosphatides which are preferentially enriched in negatively charged phospholipids such as phosphatidylglycerol, phosphatidic acid, phosphatidylserine, phosphatidylinositol, and the corresponding lysophophatides. However, the amphiphilic lipid can also be enriched in positively charged phospholipids. The compositions may optionally include one or more excipients (e.g., buffering agents, isotonicity adjusting agents, chelating agents, and antioxidants) suitable for the preparation of sterile pharmaceutical formulations for parenteral, inhalation, or topical administration.

The compositions according to the invention can be formulated into inhalation, nasal, or ocular formulations where a sterile formulation is preferred. An inhalation formulation is in the form of a sterile dispersion or suspension, wherein a composition according to the invention is a liquid for delivery of aqueous droplets comprising a glucocorticosteroid via a nebulizer to the pulmonary system (e.g. bronchial system and lungs). It is also envisioned that for inhalation, the sterile dispersion or suspension of a composition according to the invention may be utilized in combination with other liquids and excipients and optionally a propellant for delivery via a metered dose inhaler (MDI) to the pulmonary system. It is further envisioned that for inhalation, the sterile dispersion or suspension of a composition according to the invention may be utilized with other liquids or excipients and converted to a dry powder alone for delivery via a dry powder inhaler (DPI) to the pulmonary system (see e.g., US 20020102294 A1 to Bosch et al., for “Aerosols Comprising Nanoparticle Drugs”). Sterile nasal formulations can be in the form of a solution of a composition according to the invention in an appropriate liquid phase with additional excipients and stabilizers as required. Ocular formulations can be in the form of a solution of a composition according to the invention in an appropriate liquid phase with additional excipients and stabilizers as required.

Yet another aspect of the invention is directed to a pharmaceutical glucocorticosteroid nanoparticulate composition comprising a suspension for inhalation and/or a nasal spray. The pharmaceutical nanoparticulate composition comprises a therapeutically effective amount of a nanoparticulate glucocorticosteroid (e.g. budesonide, fluticasone propionate, beclomethasone dipropionate) composition together with one or more surface stabilizers and an amphiphilic lipid.

Still another aspect of the present invention is directed to a method of treating a mammal suffering from a condition for which glucocorticosteroids (e.g. budesonide, fluticasone) is indicated, comprising administering to the mammal a therapeutically effective amount of a nanoparticulate glucocorticosteroid composition of the present invention.

This invention further discloses a method of making a sterilized nanoparticulate glucocorticosteroid composition according to the invention. Such a method comprises contacting a glucocorticosteroid and at least one non-ionic surface stabilizer for a time and under conditions sufficient to provide a nanoparticulate glucocorticosteroid composition. The one or more non-ionic surface stabilizers can be contacted with a glucocorticosteroid either before, during, or after size reduction of the glucocorticosteroid. The composition is then sterilized. Prior to sterilization, at least one amphiphilic lipid is added to the composition. The amphiphilic lipid can be added either before, during, or after size reduction of the glucocorticosteroid. In addition, the dispersion can be formulated into a dry powder prior to sterilization.

The present invention is also directed to methods of treatment using the sterilized nanoparticulate glucocorticosteroid compositions of the invention.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to the surprising and unexpected discovery that nanoparticulate glucocorticosteroid compositions, comprising at least one nonionic surface stabilizer, can be successfully moist heat sterilized, when the composition to be sterilized additionally comprises at least one amphiphilic lipid. The glucocorticosteroid particles have an effective average particle size of less than about 2000 nm. As shown in the examples below, the invention is surprisingly applicable to glucocorticosteroids having different chemical structures (e.g., budesonide, beclomethasone, and fluticasone are exemplified), nonionic surface stabilizers having different structures (polysorbate-80, tyloxapol, and Lutrol F127 NF were exemplified), and amphiphilic lipids having different structures (Lecithin NF, partially purified hydrogenated lecithin (LIPOID S75-3), partially purified lecithin (LIPOID S45), distearyl phosphatidylglycerol (LIPOID PG 18:0/18:0), and dipalmityl phosphatidic acid (LIPOID PA 16:0/16:0) were exemplified). The various drugs, nonionic surface stabilizers, and amphiphilic lipids were all successfully shown to produce nanoparticulate glucocorticosteroid compositions that can be moist heat sterilized without producing significant glucocorticosteroid particle size growth.

The sterilized dispersions of nanoparticulate glucocorticosteroid can then be formulated into any suitable dosage form, such as solid, semi-solid, or liquid dosage form, including dosage forms for oral, pulmonary, nasal, parenteral, rectal, local, buccal, or topical administration. The invention is particularly useful for aqueous dosage forms which can be conducive to contamination, such as injectable, aerosol, or ocular dosage forms, or liquid dosage forms for otic administration. The sterilized dispersion can be formulated into a dry powder, such as a lyophilized powder, spray dried powder, or spray granulated powder of a nanoparticulate active agent dispersion. The dosage form can also be a controlled release formulation, solid dose fast melt formulation, aerosol formulation, lyophilized formulation, tablet, solid lozenge, capsule, powder, ocular formulation, a formulation for otic administration, or a liquid for injection.

The heat sterilization process destroys substantially all of the microbial and viral contamination in the dispersion, such as microbes, mycoplasma, yeast, viruses, and mold. The microbial contamination which is to be destroyed is generally that of bacteria,mycoplasma, yeast and mold contamination. The moist heat sterilization step: (1) results in minimal, if any, increase in glucocorticosteroid particle size on storage, (2) maintains the chemical integrity of the nanoparticulate glucocorticosteroid, and (3) shows generally acceptable impurity concentrations for the glucocorticosteroid composition following heat sterilization. The moist heat sterilization process does not significantly degrade the glucocorticosteroid or reduce the glucocorticosteroid's efficacy. The present invention enables products to meet cGMP requirements for sterile products without harming the active agent.

Surprisingly, following sterilization the dispersion of one or more nanoparticulate glucocorticosteroids exhibits unexpected overall stability, maintains the pre-sterilized physical and chemical properties, while meeting cGMP requirements for sterility. It is particularly unexpected that moist heat sterilization of the dispersion of one or more nanoparticulate glucocorticosteroids does not significantly alter the particle size of the one or more glucocorticosteroids. This is significant because if the sterilized product formed aggregates or large crystals, the dispersion would lose the benefits afforded by being formulated into a nanoparticulate glucocorticosteroid composition.

The sterile compositions of the invention, both aqueous and dry powder, are particularly useful in the treatment of respiratory-related illnesses such as asthma, emphysema, respiratory distress syndrome, chronic bronchitis, cystic fibrosis, chronic obstructive pulmonary disease, respiratory illness associated with acquired immune deficiency syndrome, and inflammatory and allergic conditions of the derma (skin) (e.g., psoriasis), eye, and ear. The formulations and method result in improved surface area coverage of the application site (e.g., lung, nasal, eye, ear, etc.) by the administered composition according to the invention.

Sterile dosage forms are particularly desirable for subjects at risk of infection, such as neonatal, pediatric, elderly, and immune compromised patients, as well as for dosage forms to be administered to areas at risk of infection (e.g., the eye, ear, mouth, lungs, nasal cavity). This need for sterile dosage forms is also demonstrated by the recent issuance by the U.S. Food and Drug Administration of guidelines requiring inhaled products to be sterile. The requirement of sterility can be problematic for formulations of nanoparticulate drugs, as heat sterilization can result in solubilization and subsequent recrystallization of the component drug particles. Furthermore, drugs which become soluble in the aqueous media may also be more labile to chemical degradation. This process results in an increase in the size distribution of the drug particles. In addition, some nanoparticulate formulations also exhibit particle aggregation following exposure to elevated temperatures for heat sterilization.

Crystal growth and particle aggregation in nanoparticulate preparations are highly undesirable for several reasons. The presence of large crystals in the nanoparticulate composition may cause undesirable side effects, especially when the preparation is in an injectable formulation. This is also true for particle aggregation. Larger particles formed by particle aggregation and recrystallization can interfere with blood flow, causing pulmonary embolism and death.

In addition, the presence of large crystals, and therefore varying particle sizes, and/or particle aggregation can change the pharmacokinetic profile of the administered drug. For oral formulations, the presence of large crystals or aggregates creates a variable bioavailability profile because smaller particles dissolve faster than the larger aggregates or larger crystal particles. A faster rate of dissolution is associated with greater bioavailability and a slower rate of dissolution is associated with a lower bioavailability. This is because bioavailability is proportional to the surface area of an administered drug and, therefore, bioavailability increases with a reduction in the particle size of the dispersed agent (see U.S. Pat. No. 5,662,833). With a composition having widely varying particle sizes, bioavailability becomes highly variable and inconsistent and dosage determinations become difficult. Moreover, because such crystal growth and particle aggregation are uncontrollable and unpredictable, the quality of the nanoparticulate compositions is inconsistent. For intravenously injected particulate formulations, the presence of large crystals or aggregates can induce an immune systems response which causes the larger particles to be transported by macrophage cells to the liver or spleen and metabolized, in addition to the embolytic effects described above.

Aggregation of nanoparticle compositions upon heating is directly related to the precipitation of the surface stabilizer at temperatures above the cloud point of the surface stabilizer. At this point, the bound surface stabilizer molecules are likely to dissociate from the nanoparticles and precipitate, leaving the nanoparticles unprotected. The unprotected nanoparticles then aggregate into clusters of particles. It was unexpectedly discovered that glucocorticosteroids, in combination with at least one nonionic surface stabilizer and at least one amphiphilic lipid, can be successfully heat sterilized, producing a sterile compositions having an effective average particle size of less than about 2000 nm, with minimal or no degradation of the glucocorticosteroid. Such particle size growth results in a loss of the pharmaceutical benefits afforded by formulating the active agent in a nanoparticulate dosage form, such as a faster onset of activity (particularly critical for treatment of asthma and allergic conditions), reduced toxicity, and a lower dosage of active agent.

A. Definitions

The present invention is described herein using several definitions, as set forth below and throughout the application.

The term “effective average particle size”, as used herein means that at least 50% of the nanoparticulate glucocorticosteroid particles have a weight average size of less than about 2000 nm, when measured by, for example, sedimentation field flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to a stable glucocorticosteroid particle connotes, but is not limited to one or more of the following parameters: (1) the glucocorticosteroid particles do not appreciably flocculate or agglomerate due to interparticle attractive forces or otherwise significantly increase in particle size over time; (2) that the glucocorticoid particles do not appreciably solubilize either during the addition of stabilizer or amphiphilic lipid, or during the subsequent moist heat treatment; (3) that the physical structure of the glucocorticosteroid particles is not altered over time, such as by conversion from an amorphous phase to a crystalline phase; (4) that the glucocorticosteroid particles are chemically stable; and/or (5) where the glucocorticosteroid has not been subject to a heating step at or above the melting point of the glucocorticosteroid in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, preferably less than about 20 mg/ml, preferably less than about 10 mg/ml, or preferably less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

B. Compositions =p Any poorly water-soluble glucocorticosteroid which is not chemically labile to moist heat treatment according to the proposed process can be used in the compositions according to the invention. Glucocorticosteroids have been shown to have a wide range of inhibitory activities against multiple cell types (e.g., mast cells, eosinophils, neutrophils, macrophages, and lymphocytes) and mediators (e.g., histamine, eicosanoids, leukotrienes and cytokines) involved in allergic and nonallergic/irritant-mediated inflammation. Corticoids affect the delayed (6 hour) response to an allergen challenge more than the histamine-associated immediate response (20 minutes).

Exemplary glucocorticosteroids include, but are not limited to, budesonide, triamcinolone, triamcinolone acetonide, mometasone, mometasone furoate, flunisolide, fluticasone, fluticasone propionate, beclomethasone, beclomethasone dipropionate, dexamethasone, fluocinolone, fluocinonide, flunisolide, flunisolide hemihydrate, mometasone furoate monohydrate, clobetasol, and combinations thereof. Preferred glucocorticosteroids are budesonide, fluticasone, triamcinolone, mometasone, beclomethasone, and combinations thereof. The amount of the glucocorticosteroid, in concentrated form or upon dilution in a pharmaceutically acceptable vehicle, typically ranges from about 0.01% to about 20%, by weight, although other glucocorticosteroid concentrations are envisioned in this invention.

In one embodiment of the invention, the glucocorticosteroid has a chemical purity of greater than 99%. In another embodiment of the invention, the glucocorticosteroid has a chemical purity of greater than 99.5%.

The sterilized glucocorticosteroid formulations of the present invention further comprise at least one non-crosslinked, non-ionic surface stabilizer. Nonionic surface stabilizers useful herein physically adhere on the surface of the nanoparticulate glucocorticosteroid but do not chemically react with the glucocorticosteroid particles or itself. Individual molecules of the surface stabilizer are preferably essentially free of intermolecular cross-linkages. As used herein, a “nonionic” surface stabilizer is a stabilizer in which the polar group of the compound is not electrically charged. Generally, the surface stabilizer has a hydrocarbon tail and a polar head whose oxygen atoms attract water molecules and make the head water soluble, but bears no ionic charge.

Exemplary non-ionic surface stabilizers include, but are not limited to, sorbitol esters, polyoxyethylene sorbitan esters, i.e., polysorbate 80, polysorbate 60; poloxamers (e.g., poloxamer 407 and Pluronic® F68, F108 and F127, which are block copolymers of ethylene oxide and propylene oxide), Polysorbates, spans, and other sorbitol esters, sorbitan oleate esters, sorbitan palmitate esters, sorbitan stearate esters, polyoxyethylene sorbitan monolaurate, polyoxyethylene sorbitan mono-oleate, glyceryl mono-oleate and glyceryl mono-laurate, as well as other surfactants containing polyethylene oxide chains and mixtures thereof, hydroxypropyl methylcellulose, hydroxypropylcellulose, polyvinylpyrrolidone (PVP), random copolymers of vinyl pyrrolidone and vinyl acetate, dextran, cholesterol, polyoxyethylene alkyl ethers (e.g., macrogol ethers such as cetomacrogol 1000), polyoxyethylene castor oil derivatives, polyethylene glycols (e.g., Carbowaxs 3550® and 934® (Union Carbide)), polyoxyethylene stearates, methylcellulose, hydroxyethylcellulose, noncrystalline cellulose, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide), p-isononylphenoxypoly-(glycidol), also known as Olin-lOG® (Olin Chemicals, Stamford, Conn.); and SA9OHCO, which is C₁₈H₃₇CH₂C(O)N(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E,, and the like. Useful nonionic surface stabilizers include polyoxyethylene sorbitan esters and in particular, polysorbate 80, commercially available as Tween 80.

The amphiphilic lipid that is incorporated into the sterilized glucocorticosteroid formulations of the present invention may be selected from one of a variety of phospholipids, provided that the composition contains some negatively charged phospholipids. Exemplary phospholipids include, but are not limited to, lecithin NF grades or synthetic phospholipids including lecithin NF, purified lecithin (LIPOID S 45), hydrogenated lecithin (LIPOID S 75-3), soy or egg lecithin phosphatides containing mixtures of anionic phophatides such as phosphatidylinositol, phosphatidylserine, phosphatidic acid, phosphatidylglycerol, the corresponding lysophosphatides, synthetic phosphatidyl glycerol (LIPOID PG 18:0/18:0), synthetic phosphatidic acid and mixtures thereof. Additional phospholipids that can be utilized in the invention include anionic phosphatides, lecithin NF, synthetic lecithin NF, synthetic phospholipids, partially purified hydrogenated lecithin, partially purified lecithin, soy lecithin phosphatides comprising anionic phophatides, egg lecithin phosphatides comprising anionic phophatides, hydrogenated soy lecithins comprising anionic phosphatides, hydrogenated egg lecithins comprising anionic phosphatides, lecithins comprising anionic phosphatides, synthetic phosphatidyl glycerol, synthetic phosphatidic acid, synthetic phosphatidyl inositol, synthetic phosphatidyl serine, phosphatidyl inositol, phosphatidyl serine, phosphatidic acid, phosphatidyl glycerol, lysophosphatidyl inositol, lysophosphatidyl serine, lysophosphatidic acid, lysophosphatidyl glycerol, distearyl phosphatidyl glycerol, distearyl phosphatidyl inositol, distearyl phosphatidyl serine, distearyl phosphatidic acid, distearyl lysophosphatidyl glycerol, distearyl lysophosphatidyl inositol, distearyl lysophosphatidyl serine, distearyl lysophosphatidic acid, dipalmityl phosphatidyl inositol, dipalmityl phosphatidyl serine, dipalmityl phosphatidic acid, dipalmityl phosphatidyl glycerol, dipalmityl lysophosphatidyl inositol, dipalmityl lysophosphatidyl serine, dipalmityl lysophosphatidic acid, dipalmityl lysophosphatidyl glycerol, and mixtures thereof In one embodiment of the invention, the amphiphilic lipid is lecithin, and the lecithin comprises less than 90% phosphatidylcholine. In yet another embodiment of the invention, the amphiphilic lipid is lecithin, and the lecithin is comprised substantially of hydrogenated phosphatidylcholine and the remaining composition composed of mainly hydrogenated anionic phosphatides.

The sterilized glucocorticosteroid formulations of the present invention may additionally comprise a chelating agent, such as ethylenediamine tetraacetic acid (EDTA) or ethylene glycol-bis(beta-aminoethyl ether)-N,N,N′,N′-tetraacetic acid (EGTA), which is added to the formulation just prior to the sterilization step. Preferably, the amount of EDTA or EGTA added to the glucocorticosteroid formulation is dependent on the amount of amphiphilic lipid added as a surface stabilizer. The greater the amount of the amphiphilic lipid added, the greater the amount of EDTA or EGTA is added and conversely, vice versa—the less amphiphilic lipid added, the less EDTA or EGTA added. Thus, in one embodiment of the invention, the composition can comprise a sodium salt or calcium salt of EDTA or EGTA, or a combination thereof. In another embodiment of the invention, the amount of sodium salt and/or calcium salt of EDTA or EGTA can range from about 0.0001% to about 5%, from about 0.001 to about 1%, and from about 0.01% to about 0.1%.

The compositions of the invention can be formulated into any suitable dosage form. For example, the compositions of the invention can be formulated for injectable, otic, oral, rectal, pulmonary, opthalmic, colonic, parenteral, intracisternal, intravaginal, intraperitoneal, local, buccal, nasal, or topical administration; the compositions of the invention can be formulated into a powder, lyophilized powder, spray dried powder, spray granulated powder, solid lozenge, capsule, tablet, pill, granule, liquid dispersion, gel, aerosol, ointment, or cream; the compositions of the invention can be formulated into a dosage form such as a controlled release formulation, solid dose fast melt formulation, controlled release formulations, fast melt formulations, lyophilized formulations, delayed release formulations, extended release formulations, pulsatile release formulations, and mixed immediate release and controlled release formulations; or any combination thereof Dosage forms that are preferably sterile include, but are not limited to, aerosols for nasal or pulmonary delivery, injectable, and opthalmic dosage forms.

### 1. Aqueous Aerosols

One embodiment of a nanoparticulate glucocorticosteroid dispersion for nasal, pulmonary (upper lung), lung (deep lung), mouth, ocular, or otic delivery is an aerosol (e.g., nasal aerosols, lingual (mouth) aerosols, or inhalation aerosols). Aqueous formulations of the present invention consist of colloidal dispersions of poorly water-soluble nanoparticulate glucocorticosteroid compositions in an aqueous vehicle, which is aerosolized using air-jet or ultrasonic nebulizers. The advantages of the use of such aqueous aerosols can best be understood by comparing the sizes of nanoparticulate and conventional micronized glucocorticosteroid compositions according to the invention with the sizes of liquid droplets produced by conventional nebulizers. Conventional micronized material is generally about 2 to about 5 microns or more in diameter and is approximately the same size as the liquid droplet size produced by medical nebulizers. In contrast, nanoparticulate glucocorticosteroid compositions having a size of 2 microns or less are equivalent or smaller than the droplets in such an aerosol. Thus, aerosols containing nanoparticulate glucocorticosteroid compositions according to the invention improve drug delivery efficiency. Such aerosols can also contain a higher number of nanoparticles per unit dose, resulting in each aerosolized glucocorticosteroid droplet containing active compositions according to the invention.

Thus, with administration of the same dosages of compositions according to the invention, more lung or nasal cavity surface area is covered by the aerosol formulation containing a nanoparticulate glucocorticosteroid compositions.

Another advantage of the use of these aqueous aerosols is that they permit poorly water-soluble compositions according to the invention to be delivered to the deep lung via an aqueous formulation. Conventional micronized drug substances are too large to reach the peripheral lung regardless of the size of the droplets produced by the nebulizer. The aqueous aerosols comprised of compositions according to the invention permit nebulizers which generate very small (about 0.5 to about 2 microns) aqueous droplets to deliver water-insoluble compositions according to the invention in the form of nanoparticles to the alveoli. One example of such devices is the Circular™ aerosol (Westmed Corp., Tucson, Ariz.).

Yet another advantage of the aqueous glucocorticosteroid aerosols is that ultrasonic nebulizers can be used to deliver a poorly water-soluble composition according to the invention to the lung. Unlike conventional micronized compositions according to the invention, compositions according to the invention in the form of nanoparticles are readily aerosolized and show good in vitro deposition characteristics. A specific advantage of these aqueous glucocorticosteroid aerosols is that they permit poorly water-soluble glucocorticosteroid compositions to be aerosolized by ultrasonic nebulizers which require nanoparticles comprised of compositions according to the invention to pass through very fine orifices to control the size of the aerosolized droplets. While conventional drug material would be expected to occlude the pores, such nanoparticulates are much smaller and can pass through the pores without difficulty.

For aqueous aerosol formulations, a nanoparticulate glucocorticosteroid composition according to the invention is present at a concentration of about 0.001 mg/mL up to about 600 mg/mL. In other embodiments of the invention, the glucocorticosteroid can be present at a concentration of about 0.025 mg/mL up to about 3 mg/mL; about 10 mg/mL or more, about 100 mg/mL or more, about 200 mg/mL or more, about 400 mg/mL or more, or about 600 mg/mL. Dry powder aerosols of the glucocorticosteroid compositions of the invention are also encompassed by the invention. For dry powder aerosol formulations, compositions according to the invention are present at a concentration of about 0.001 mg/g up to about 990 mg/g, depending on the desired dosage. Concentrated nanoparticulate aerosols, defined as containing a composition according to the invention at a concentration of about 0.025 mg/mL up to about 3 mg/mL, or about 10 mg/mL up to about 600 mg/mL for aqueous glucocorticosteroid aerosol formulations, and about 0.025 mg/g up to about 3 mg/g, or about 10 mg/g up to about 990 mg/g for dry powder aerosol formulations, are specifically encompassed by the present invention. Such formulations provide effective delivery to appropriate areas of the mouth, lung or nasal cavities in short administration times, i.e., less than about 15 seconds as compared to administration times of up to 4 to 20 minutes as found in conventional pulmonary nebulizer therapies. In other embodiments of the invention, the aerosol can be administered in a time of from about 10 seconds up to about 30 minutes, from about 10 seconds up to about 25 minutes, from about 10 seconds up to about 20 minutes, from about 10 seconds up to about 15 minutes, from about 10 seconds up to about 10 minutes, from about 10 seconds up to about 9 minutes, from about 10 seconds up to about 8 minutes, from about 10 seconds up to about 7 minutes, from about 10 seconds up to about 6 minutes, from about 10 seconds up to about 5 minutes, from about 10 seconds up to about 4 minutes, from about 10 seconds up to about 3 minutes, from about 10 seconds up to about 2 minutes, from about 10 seconds up to about 1 minute. In yet other embodiments of the invention, the aerosol of the invention can be administered in a time of about 10 seconds or greater, about 15 seconds or greater, about 20 seconds or greater, about 25 seconds or greater, about 30 seconds or greater, about 35 seconds or greater, about 40 seconds or greater, about 45 seconds or greater, about 50 seconds or greater, or about 55 seconds or greater, or any combination thereof, such as from about 20 seconds up to about 8 minutes.

In one embodiment of the invention the droplets of the aerosol have a mass median aerodynamic diameter (MMAD) less than or equal to about 100 microns. In other embodiments of the invention, the droplets of the aerosol have a mass median aerodynamic diameter (MMAD) of (1) from about 0. 1 to about 10 microns; (2) from about 2 to about 6 microns; (3) less than about 2 microns; (4) from about 5 to about 100 microns; or (5) from about 30 to about 60 microns. In another embodiment of the invention, essentially each droplet of the aqueous aerosol comprises at least one nanoparticulate glucocorticosteroid particle.

### 2. Dry Powder Aerosol Formulations

A dry powder inhalation formulation can be made by spray-drying an aqueous nanoparticle glucocorticosteroid dispersion of a composition according to the invention. Alternatively, dry powders containing a nanoparticulate composition according to the invention can be made by freeze-drying the dispersions of the nanoparticles. Combinations of the spray-dried and freeze-dried nanoparticulate powders can be used in DPIs and pMDIs. For dry powder aerosol formulations, a nanoparticulate composition according to the invention may be present at a concentration of about 0.025 mg/g up to about 990 mg/g.

Dry powder inhalers (DPIs), which involve de-aggregation and aerosol formulation of dry powders, normally rely upon a burst of inspired air that is drawn through the unit to deliver a drug dosage. Such devices are described in, for example, U.S. Pat. No. 4,807,814, the entire contents of which is incorporated herein by reference, which is directed to a pneumatic powder ejector having a suction stage and an injection stage; SU 628930 (Abstract), describing a hand-held powder disperser having an axial air flow tube; Fox et al., Powder and Bulk Engineering, pages 33-36 (March 1988), describing a venturi eductor having an axial air inlet tube upstream of a venturi restriction; EP 347 779, describing a hand-held powder disperser having a collapsible expansion chamber, and U.S. Pat. No. 5,785,049, the entire content of which is incorporated herein by reference, directed to dry powder delivery devices for drugs.

A dry powder inhalation formulation can also be delivered by means of an aerosol formulation. The powders may consist of inhalable aggregates of nanoparticulate compositions according to the invention, or of inhalable particles of a diluent which contains at least one embedded composition according to the invention. Powders containing a nanoparticulate composition according to the invention can be prepared from aqueous dispersions of nanoparticles by removing the water by spray-drying or lyophilization (freeze drying). Spray-drying is less time consuming and less expensive than freeze-drying, and therefore more cost-effective.

Dry powder aerosol delivery devices must be able to accurately, precisely, and repeatably deliver the intended amount of a composition according to the invention. Moreover, such devices must be able to fully disperse the dry powder into individual particles of a respirable size. Conventional micronized drug particles of 2-3 microns in diameter are often difficult to meter and disperse in small quantities because of the electrostatic cohesive forces inherent in such powders. These difficulties can lead to loss of drug substance to the delivery device as well as incomplete powder dispersion and sub-optimal delivery to the lung. Many drug compounds are intended for deep lung delivery and systemic absorption. Since the average particle sizes of conventionally prepared dry powders are usually in the range of 2-3 microns, the fraction of material which actually reaches the alveolar region may be quite small. Thus, delivery of micronized dry powders to the lung, especially the alveolar region, is generally very inefficient because of the properties of the powders themselves.

The dry powder aerosols which contain nanoparticulate compositions according to the invention can be made smaller than comparable micronized drug substance and, therefore, are appropriate for efficient delivery to the deep lung. Moreover, aggregates of nanoparticulate compositions according to the invention are spherical in geometry and have good flow properties, thereby aiding in dose metering and deposition of the administered composition in the lung or nasal cavities.

Dry nanoparticulate compositions can be used in both DPIs and pMDIs. (Within the context of the present invention, “dry” refers to a composition having less than about 5% water.). Nanoparticulate aerosol formulations are described in U.S. Pat. No. 6,811,767 to Bosch et al., which is specifically incorporated herein by reference.

Nasal formulations can be in the form of a solution of a composition according to the invention in an appropriate solvent or a dispersion or suspension of a composition according to the invention in a liquid phase and a stabilizer and a dry powder. A solution is comprised of a composition according to the invention and an appropriate solvent and optionally one or more co-solvents. Water is the typical solvent. However, composition according to the invention may not be soluble in water alone in which case one or more co-solvents may have to be employed in order to form a solution. Suitable co-solvents include, but are not limited to, short-chained alcohols, and in particular, ethanol.

Nasal formulations can also be in the form of a dispersion or suspension. In these types of formulations, a composition according to the invention can be in the form of a glucocorticosteroid nanoparticle which is dispersed or suspended in water with or without one or more suspending agents. Inhalation therapies, (i.e., dose inhalers) containing nanoparticulate glucocorticosteroid compositions according to the invention and pMDIs (pressured metered dose inhalers) can comprise either the discrete nanoparticles and surface stabilizer, aggregates of the nanoparticles and surface stabilizer, or motive diluent particles containing the embedded nanoparticles or solutions of the drugs or combinations in solvents and/or propellants. pMDIs can be used for targeting the nasal cavity, the conducting airways of the lung or the alveoli. Compared to conventional formulations, the present invention affords increased delivery to the deep lung regions because the inhaled nanoparticles are smaller than conventional micronized material (<2 microns) and are distributed over a larger mucosal or alveolar surface area as compared to micronized drugs.

### a. Spray-Dried Powders Containing Glucocorticosteroid Nanoparticles

Powders comprising a nanoparticulate glucocorticosteroid composition according to the invention can be made by spray-drying aqueous dispersions of a nanoparticulate composition and a surface stabilizer to form a dry powder which consists of an aggregated nanoparticulate composition according to the invention. The aggregates can have a size of about 1 to about 2 microns which is suitable for deep lung delivery. The aggregate particle size can be increased to target alternative delivery sites, such as the upper bronchial region or nasal mucosa by increasing the concentration of a composition according to the invention in the spray-dried dispersion or by increasing the droplet size generated by the spray dryer.

Alternatively, the aqueous dispersion of a nanoparticulate glucocorticosteroid composition according to the invention and the surface stabilizer(s) can contain a dissolved diluent such as lactose or mannitol which, when spray dried, forms inhalable diluent particles, each of which contains at least one embedded glucocorticosteroid nanoparticle, nonionic surface stabilizer, and amphiphilic lipid according to the invention. The diluent particles with an embedded glucocorticosteroid nanoparticles can have a particle size of about 1 to about 2 microns, suitable for deep lung delivery. In addition, the diluent particle size can be increased to target alternate delivery sites, such as the upper bronchial region or nasal mucosa by increasing the concentration of dissolved diluent in the aqueous dispersion prior to spray drying, or by increasing the droplet size generated by the spray dryer.

Spray-dried powders can be used in DPIs or pMDIs, either alone or combined with freeze-dried nanoparticulate powder. In addition, spray-dried powders containing a nanoparticulate composition according to the invention can be reconstituted and used in either jet or ultrasonic nebulizers to generate aqueous dispersions having respirable droplet sizes, where each droplet contains at least one nanoparticulate composition according to the invention. Concentrated nanoparticulate dispersions may also be used in these aspects of the invention.

### b. Freeze-Dried Powders Containing a Nanoparticulate Composition According to the Invention

Nanoparticulate glucocorticosteroid compositions according to the invention in the form of nanoparticle glucocorticosteroid dispersions can also be freeze-dried to obtain powders suitable for nasal or pulmonary delivery. Such powders may contain aggregated nanoparticulate glucocorticosteroid compositions according to the invention having at least one nonionic surface stabilizer and at least one amphiphilic lipid. Such aggregates may have sizes within a respirable range, i.e., about 2 to about 5 microns. Larger aggregate particle sizes can be obtained for targeting alternate delivery sites, such as the nasal mucosa.

Freeze dried powders of the appropriate particle size can also be obtained by freeze drying aqueous dispersions of a composition according to the invention, which additionally contain a dissolved diluent such as lactose or mannitol. In these instances the freeze dried powders consist of respirable particles of diluent, each of which contains at least one embedded nanoparticulate composition according to the invention.

Freeze-dried powders can be used in DPIs or pMDIs, either alone or combined with spray-dried nanoparticulate powder. In addition, freeze-dried powders containing a nanoparticulate composition according to the invention can be reconstituted and used in either jet or ultrasonic nebulizers to generate aqueous dispersions having respirable droplet sizes, where each droplet contains at least one nanoparticulate composition according to the invention. Concentrated nanoparticulate dispersions may also be used in these aspects of the invention.

### 3. Particle Size

The compositions of the present invention contain nanoparticulate glucocorticosteroid particles which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the glucocorticosteroid particles have a particle size of less than the effective average, by weight, i.e., less than about 2000 nm, 1900 nm, 1800 nm, etc. (as listed above), when measured by the above-noted techniques. Preferably, at least about 70%, at least about 80%, at least about 90%, at least about 95%, or at least about 99% of the glucocorticosteroid particles, by weight, have a particle size of less than the effective average, i.e., less than about 2000 nm, 1900 nm, 1800 nm, 1700 nm, etc.

In the present invention, the value for D50 of a nanoparticulate glucocorticosteroid composition is the particle size below which 50% of the glucocorticosteroid particles fall, by weight. Similarly, D90 is the particle size below which 90% of the glucocorticosteroid particles fall, by weight, and D99 is the particle size below which 99% of the glucocorticosteroid particles fall, by weight.

### 4. Concentration of the Glucocorticosteroid, Nonionic Surface Stabilizer, and Amphiphilic Lipid

The relative amounts of a glucocorticosteroid, one or more nonionic surface stabilizers, and at least one amphiphilic lipid can vary widely. The optimal amount of the individual components can depend, for example, upon the particular glucocorticosteroid selected, the particular nonionic surface stabilizer selected, the particular amphiphilic lipid selected, the hydrophilic lipophilic balance (HLB), melting point, and the surface tension of water solutions of the nonionic surface stabilizer, etc.

In one embodiment, the concentration of the glucocorticosteroid can vary from about 99.5% to about 0.001%, from about 95% to about 0.1%, or from about 90% to about 0.5%, by weight, based on the total combined weight of the glucocorticosteroid, at least one nonionic surface stabilizer, and at least one amphiphilic lipid, not including other excipients.

In another embodiment, the concentration of the at least one non-ionic surface stabilizer can vary from about 0.01% to about 99%, from about 0. 1% to about 50%, and from about 1% to about 10%, by weight, based on the total combined weight of the glucocorticosteroid, at least one nonionic surface stabilizer, and at least one amphiphilic lipid, not including other excipients.

In another embodiment, the concentration of the at least one amphiphilic lipid can vary from about 0.01% to about 99%, from about 0.1% to about 50%, and from about 1% to about 10%, by weight, by weight, based on the total combined weight of the glucocorticosteroid, at least one nonionic surface stabilizer, and at least one amphiphilic lipid, not including other excipients.

In an exemplary embodiment of the invention, the nanoparticulate glucocorticosteroid compositions comprise a glucocorticosteroid concentration of from about 10 to 30% w/w in contact with a nonionic surface stabilizer which comprises from about 5 to 10% of the total glucocorticosteroid concentration.

### 5. Combination Compositions

The dispersions to be sterilized can comprise multiple glucocorticosteroids, compositions of one or more glucocorticosteroids having multiple particle sizes, or a combination thereof. For example, a dispersion can comprise: (1) nanoparticulate glucocorticosteroid A and nanoparticulate glucocorticosteroid B; (2) nanoparticulate glucocorticosteroid A and microparticulate glucocorticosteroid A; (3) nanoparticulate glucocorticosteroid A and microparticulate glucocorticosteroid B; (3) nanoparticulate glucocorticosteroid A having an effective average particle size of 250 nm and nanoparticulate glucocorticosteroid A having an effective average particle size of 800 nm, or combinations thereof.

### a. Compositions Comprising Microparticulate Active Agents

Sterilized microparticulate glucocorticosteroid particles can be combined with the sterilized dispersion of one or more nanoparticulate glucocorticosteroid particles, either prior or subsequent to sterilization, to provide for a sustained or controlled release composition. Such sterilized microparticulate glucocorticosteroid particles can also be combined with a sterilized dispersion which has been processed into a powder or other dry dosage form.

The combination of very small glucocorticosteroid particles, i.e., nanoparticulate glucocorticosteroid particles, in combination with larger active agent particles, i.e., micronized glucocorticosteroid particles, can enable obtaining the simultaneous presentation of immediate-release (IR) and controlled-release (CR) glucocorticosteroid components. The micronized glucocorticosteroid particles and nanoparticulate glucocorticosteroid particles can be the same glucocorticosteroid or different glucocorticosteroid.

For the purposes of this invention, “nanoparticulate” active agents have an effective average particle size of less than about 2 microns and micronized active agents have an effective average particle size of greater than about 2 microns. The micronized active agent particles can be sterilized simultaneously with the nanoparticulate active agent particles or in a separate process using a suitable sterilization method.

The nanoparticulate glucocorticosteroid particles, representing the IR component, afford rapid in vivo dissolution, owing to their small size and attendant large specific surface. The micronized glucocorticosteroid particles, representing the CR component, afford slower in vivo dissolution, owing to a comparatively large particle size and small attendant specific surface.

IR and CR components representing a wide range of in vivo dissolution rates (and hence, in vivo input rates for absorption) can be engineered through precise control of glucocorticosteroid particle size. Thus, the compositions can comprise a mixture of nanoparticulate glucocorticosteroid particles, wherein each population of particles has a defined size correlating with a precise release rate, and the compositions can comprise a mixture of microparticulate glucocorticosteroid particles, wherein each population of particles has a defined size correlating with a precise release rate.

### b. Compositions Comprising Multiple Nanoparticulate Particle Sizes

In yet another embodiment of the invention, a dispersion of a first nanoparticulate glucocorticosteroid providing a desired pharmacokinetic profile combined with at least one other dispersion of a nanoparticulate glucocorticosteroid that generates a desired different pharmacokinetic profile. More than two dispersions of nanoparticulate glucocorticosteroid can be combined. While the first glucocorticosteroid dispersion has a nanoparticulate particle size, the additional one or more glucocorticosteroid can be nanoparticulate, solubilized, or have a conventional microparticulate particle size.

The second, third, fourth, etc., glucocorticosteroid dispersions can differ from the first, and from each other, for example: (1) in the effective average particle sizes of the glucocorticosteroid; or (2) in the dosage of the glucocorticosteroid.

Preferably where co-administration of a “fast-acting” formulation and a “longer-lasting” formulation is desired, the two formulations are combined within a single composition, for example a dual-release composition.

### 6. Glucocorticosteroid Compositions Used in Conjunction with Other Active Agents

The glucocorticosteroid compositions of the invention can additionally comprise one or more compounds useful in treating asthma, allergic conjunctivitis and seasonal allergic rhinitis, and other inflammatory and allergic conditions for which glucocorticosteroids are conventionally used. The compositions of the invention can be co-formulated with such other active agents, or the compositions of the invention can be co-administered or sequentially administered in conjunction with such active agents.

Examples of active agents useful in treating asthma or allergic conditions, and that can be used in conjunction with the compositions of the invention, include but are not limited to long-acting beta-agonists, such as salmeterol (Serevent®) and formoterol (Foradil®); leukotriene modifiers, such as monoleukast (Singulair®), zafirlukast (Accolate®), and zileuton (Zyflo®); theophylline (Aerolate®, Choledyl®, Elixophyllin®, Quibron®), Slo-bid®, Theochron®, T-Phyl®, and Uniphyl®); nedocromil (Tilade®); cromolyn (Intal®); short-acting beta-agonists (also known as “bronchodilators”), such as albuterol (Airet®, Proventil®, and Ventolin®), levalbuterol (Xopenex®), bitolterol (Tornalate®), pirbuterol (Maxair®), and terbutaline (Brethaire®); ipratropium bromide (Atrovent®); prednisone (Deltasone® and Orasone®); prednisolone (Prelone® and Pediapred®); and methylprednisolone (Medrol®).

### 7. Additional Surface Stabilizers

In one embodiment of the invention, the compositions can also include one or more ionic, anionic, or zwitterionic surface stabilizers. If such surface stabilizers are utilized in a composition according to the invention, they are preferably added after moist heat sterilization of the composition. Exemplary useful ionic, anionic, or zwitterionic surface stabilizers include, but are not limited to, known organic and inorganic pharmaceutical excipients. Such excipients include various polymers, low molecular weight oligomers, natural products, and surfactants. Combinations of more than one surface stabilizer can be used in the invention.

Representative examples of ionic, cationic, anionic, or zwitterionic surface stabilizers include, but are not limited to, sodium lauryl sulfate, dioctylsulfosuccinate, gelatin, casein, gum acacia, tragacanth, stearic acid, benzalkonium chloride, calcium stearate, glycerol monostearate, cetostearyl alcohol, cetomacrogol emulsifying wax, colloidal silicon dioxide, phosphates, carboxymethylcellulose calcium, carboxymethylcellulose sodium, hydroxypropylmethylcellulose phthalate, magnesium aluminium silicate, triethanolamine, poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate ( Dow); Crodestas F-110®, which is a mixture of sucrose stearate and sucrose distearate (Croda Inc.); Crodestas SL-40® (Croda, Inc.); lysozyme, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate. Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)₄ ammonium chloride or bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Particularly preferred nonpolymeric primary stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is a alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference. The surface stabilizers are commercially available and/or can be prepared by techniques known in the art.

### 8. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicifized microcrystalline cellulose (SMCC).

Suitable lubricants, including agents that act on the flowability of the powder to be compressed, are colloidal silicon dioxide, such as Aerosil® 200; talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel®PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents, or vehicles include water, ethanol, sodium chloride, Ringer's solution, lactated Ringer's solution, stabilizer solutions, tonicity enhancers (sucrose, dextrose, mannitol, etc.) polyols (propyleneglycol, polyethylene-glycol, glycerol, and the like), suitable mixtures thereof, vegetable oils (such as olive oil) and injectable organic esters such as ethyl oleate. Suitable fluids are referenced in Remington's Pharmaceutical Sciences, 17^{th} edition, published by Mack Publishing Co., page 1543.

### D. Methods of Making the Compositions of the Invention

In another aspect of the invention there is provided a method of preparing the nanoparticulate glucocorticosteroid formulations of the invention. The method comprises of one of the following methods: attrition, precipitation, evaporation, or combinations of these. Exemplary methods of making nanoparticulate compositions are described in U.S. Pat. No. 5,145,684. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

Following milling, homogenization, precipitation, etc., the resultant nanoparticulate glucocorticosteroid composition can be sterilized and then utilized in a suitable dosage form for administration.

Preferably, the dispersion media used for the size reduction process is aqueous. However, any media in which the glucocorticosteroid is poorly soluble and dispersible can be used as a dispersion media. Non-aqueous examples of dispersion media include, but are not limited to, aqueous salt solutions, safflower oil and solvents such as ethanol, t-butanol, hexane, and glycol.

Effective methods of providing mechanical force for particle size reduction of glucocorticosteroids include ball milling, media milling, and homogenization, for example, with a Microfluidizer® (Microfluidics Corp.). Ball milling is a low energy milling process that uses milling media, drug, stabilizer, and liquid. The materials are placed in a milling vessel that is rotated at optimal speed such that the media cascades and reduces the drug particle size by impaction. The media used must have a high density as the energy for the particle reduction is provided by gravity and the mass of the attrition media.

### 1. Milling of Glucocorticosteroids for Particle Size Reduction

For milling, particles of a composition according to the invention are dispersed in a liquid dispersion media in which the particles are poorly soluble and mechanical means is applied in the presence of grinding media to reduce the particle size of the composition according to the invention to the desired effective average particle size. The particles can be reduced in size in the presence of one or more nonionic surface stabilizers. Alternatively, the particles can be contacted with one or more nonionic surface stabilizers after attrition. Other compounds, such as a diluent, can be added to the composition during the size reduction process. Dispersions can be manufactured continuously or in a batch mode.

Media milling is a high energy milling process. Drug, stabilizer, and liquid are placed in a reservoir and recirculated in a chamber containing media and a rotating shaft/impeller. The rotating shaft agitates the media which subjects the drug to impaction and sheer forces, thereby reducing the drug particle size.

For milling, a composition according to the invention can be added to a liquid media in which it is essentially insoluble to form a premix. The concentration of the composition according to the invention in the liquid media can vary from about 5 to about 60%, from about 15 to about 50% (w/v), and from about 20 to about 40%. The nonionic surface stabilizer can be present in the premix or it can be added to the drug dispersion following particle size reduction. The concentration of the nonionic surface stabilizer can vary from about 0. 1 to about 50%, from about 0.5 to about 20%, and from about 1 to about 10%, by weight.

The premix can be used directly by subjecting it to mechanical means to reduce the average particle size of the composition according to the invention in the dispersion to less than about 2000 nm. It is preferred that the premix be used directly when a ball mill is used for attrition. Alternatively, a composition according to the invention and the surface stabilizer can be dispersed in the liquid media using suitable agitation, e.g., a Cowles type mixer, until a homogeneous dispersion is observed in which there are no large agglomerates visible to the naked eye. It is preferred that the premix be subjected to such a premilling dispersion step when a recirculating media mill is used for attrition.

The mechanical means applied to reduce the particle size of a composition according to the invention conveniently can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1,000 centipoise, and for ball milling the apparent viscosity of the premix is preferably from about 1 up to about 100 centipoise. Such ranges tend to afford an optimal balance between efficient particle size reduction and media erosion.

The attrition time can vary widely and depends primarily upon the particular mechanical means and processing conditions selected. For ball mills, processing times of up to five days or longer may be required. Alternatively, processing times of less than one day (residence times of one minute up to several hours) are possible with the use of a high shear media mill.

### 2. Non-Aqueous Non-Pressurized Milling System

In a non-aqueous, non-pressurized milling system, a non-aqueous liquid having a vapor pressure of about 1 atm or less at room temperature and in which the composition according to the invention is essentially insoluble is used as a wet milling media to make a nanoparticulate composition according to the invention. In such a process, a slurry comprised of the composition according to the invention is milled in a non-aqueous media to generate a nanoparticulate composition according to the invention, followed by moist heat sterilization. Examples of suitable non-aqueous media include ethanol, trichloromonofluoromethane, (CFC-11), and dichlorotetrafluoroethane (CFC-114). An advantage of using CFC-11 is that it can be handled at only marginally cool room temperatures, whereas CFC-114 requires more controlled conditions to avoid evaporation. Upon completion of milling the composition may be sterilized and the liquid media may be removed and recovered under vacuum or heating, resulting in a dry nanoparticulate composition comprised of a composition according to the invention. Alternatively, following removal of the liquid media the dry composition can be sterilized. The dry composition may then be filled into a suitable container and charged with a final propellant. Exemplary final product propellants, which ideally do not contain chlorinated hydrocarbons, include HFA-134a (tetrafluoroethane) and HFA-227 (heptafluoropropane). While non-chlorinated propellants may be preferred for environmental reasons, chlorinated propellants may also be used in this aspect of the invention.

In a non-aqueous, pressurized milling system, a non-aqueous liquid media having a vapor pressure significantly greater than 1 atm at room temperature is used in the milling process to make a composition comprised of a nanoparticulate composition according to the invention. The composition is then sterilized. If the milling media is a suitable halogenated hydrocarbon propellant, the resultant dispersion may be filled directly into a suitable pMDI container. Alternately, the milling media can be removed and recovered under vacuum or heating to yield a dry composition comprised of a nanoparticulate composition according to the invention. This composition can then be sterilized, filled into an appropriate container, and charged with a suitable propellant for use in a pMDI.

### 3. Grinding Media

The grinding media can comprise particles that are preferably substantially spherical in shape, e.g., beads, consisting essentially of polymeric resin. Alternatively, the grinding media can comprise a core having a coating of a polymeric resin adhered thereon.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin™ (E.I. du Pont de Nemours and Co.); vinyl chloride polymers and copolymers; polyurethanes; polyamides; poly(tetrafluoroethylenes), e.g., Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably from about 0.03 to about 1 mm in size.

The polymeric resin can have a density from about 0.8 to about 3.0 g/cm³.

In a preferred grinding process the particles are made continuously. Such a method comprises continuously introducing a composition according to the invention into a milling chamber, contacting the composition according to the invention with grinding media while in the chamber to reduce the particle size of the composition according to the invention, and continuously removing the nanoparticulate composition according to the invention nanoparticles from the milling chamber.

The grinding media is separated from the milled nanoparticulate composition according to the invention nanoparticles using conventional separation techniques, in a secondary process such as by simple filtration, sieving through a mesh filter or screen, and the like. Other separation techniques such as centrifugation may also be employed.

### 4. Homogenization of Glucocorticosteroids for Particle Size Reduction

Homogenization is a technique that does not use milling media. Drug, nonionic surface stabilizer, and liquid (or drug and liquid with the nonionic surface stabilizer added after particle size reduction) constitute a process stream propelled into a process zone, which in the Microfluidizer® is called the Interaction Chamber. The product to be treated is inducted into the pump, and then forced out. The priming valve of the Microfluidizer® purges air out of the pump. Once the pump is filled with product, the priming valve is closed and the product is forced through the interaction chamber. The geometry of the interaction chamber produces powerful forces of sheer, impact, and cavitation which are responsible for particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size, which is then suitable for sterilization. The Microfluidizer® also provides a heat exchanger to allow cooling of the product. U.S. Pat. No. 5,510,118, which is specifically incorporated by reference, refers to a process using a Microfluidizer® resulting in nanoparticulate particles.

### 5. Precipitation to Obtain Nanoparticulate Compositions According to the Invention

Another method of forming the desired nanoparticle glucocorticosteroid dispersion is by microprecipitation. This is a method of preparing stable dispersions of nanoparticulate particles of the composition according to the invention in the presence of one or more nonionic surface stabilizers and one or more colloid stability enhancing surface active agents free of any trace toxic solvents or solubilized heavy metal impurities. Such a method comprises, for example, (1) dissolving the composition according to the invention, in a suitable solvent with mixing; (2) adding the formulation from step (1) with mixing to a solution comprising at least one nonionic surface stabilizer to form a clear solution; and (3) precipitating the formulation from step (2) with mixing using an appropriate nonsolvent. The method can be followed by removal of any formed salt, if present, by dialysis or diafiltration and concentration of the dispersion by conventional means. The resultant nanoparticulate composition according to the invention nanoparticle dispersion can be sterilized and then utilized, for example, in liquid nebulizers or processed to form a dry powder for use in a DPI or pMDI.

### 6. Supercritical Fluid Methods of Making Nanoparticles

Nanoparticulate compositions can also be made in methods utilizing supercritical fluids. In such a method, a glucocorticosteroid is dissolved in a solution or vehicle which can also contain at least one nonionic surface stabilizer. The solution and a supercritical fluid are then co-introduced into a particle formation vessel. If a nonionic surface stabilizer was not previously added to the vehicle, it can be added to the particle formation vessel The temperature and pressure are controlled, such that dispersion and extraction of the vehicle occur substantially simultaneously by the action of the supercritical fluid. Chemicals described as being useful as supercritical fluids include carbon dioxide, nitrous oxide, sulphur hexafluoride, xenon, ethylene, chlorotrifluoromethane, ethane, and trifluoromethane.

Examples of known supercritical methods of making nanoparticles include International Patent Application No. WO 97/144407 to Pace et al., published on Apr. 24, 1997, which refers to particles of water insoluble biologically active compounds with an average size of 100 nm to 300 nm prepared by dissolving the compound in a solution and then spraying the solution into compressed gas, liquid, or supercritical fluid in the presence of appropriate surface stabilizers. For the present invention, the surface stabilizer utilized is a nonionic surface stabilizer.

Similarly, U.S. Pat. No. 6,406,718 to Cooper et al. describes a method for forming a particulate fluticasone propionate product comprising the co-introduction of a supercritical fluid and a vehicle containing at least fluticasone propionate in solution or suspension into a particle formation vessel, the temperature and pressure in which are controlled, such that dispersion and extraction of the vehicle occur substantially simultaneously by the action of the supercritical fluid. Chemicals described as being useful as supercritical fluids include carbon dioxide, nitrous oxide, sulphur hexafluoride, xenon, ethylene, chlorotrifluoromethane, ethane, and trifluoromethane. The supercritical fluid may optionally contain one or more modifiers, such as methanol, ethanol, ethyl acetate, acetone, acetonitrile or any mixture thereof. A supercritical fluid modifier (or co-solvent) is a chemical which, when added to a supercritical fluid, changes the intrinsic properties of the supercritical fluid in or around the critical point. According to Cooper et al., the fluticasone propionate particles produced using supercritical fluids have a particle size range of 1 to 10 microns, preferably 1 to 5 microns.

### 7. Exemplary Methods of Making the Glucocorticosteroid Compositions

In an exemplary method, the nanoparticulate composition comprising a glucocorticosteroid and a nonionic surface stabilizer is diluted with water to about 5 to 20% (w/w) glucocorticosteroid and about 0.25% to about 2.0% (w/w) nonionic surface stabilizer. Lecithin phosphatides which contain some anionic phosphatides are added to the diluted nanoparticulate glucocorticosteroid composition at a concentration which represents less than about 1% to less than about 5% (w/w) of the glucocorticosteroid concentration. Thus about 0.05% to about 1% (w/w) lecithin phosphatides generate glucocorticosteroid nanoparticles.

Additional excipients or components useful in chemical protection of the glucocorticosteroid (e.g. EDTA, antioxidant, nitrogen) during the heat sterilization process may also be added to the nanoparticulate glucocorticosteroid composition.

The nanoparticulate glucocorticosteroid composition is then subjected to steam heat autoclaving at temperatures from about 116° C. to about 130° C., optimally at the temperature of 121° C. for a time period appropriate to achieve a sterilizing cycle against potential microbial, yeast, and mold contamination.

The sterilized nanoparticulate glucocorticosteroid composition is diluted and further compounded under aseptic conditions to achieve an acceptable sterile pharmaceutical composition suitable for the treatment of inflammatory and allergic conditions, such as for the treatment of inflammatory and allergic conditions of the pulmonary, nasal, ocular, and otic systems. The additional compounding may include excipients such as buffers and tonicity agents.

Exemplary final pharmaceutical compositions can consist of glucocorticosteroid at a concentration of about 0.00125% to about 0.05%, nonionic surface stabilizer at a concentration of about 0.000625% to about 0.005%, and an amphiphilic lipid at a concentration of about 0.0000125% to about 0.0025%. The final pharmaceutical composition following steam heat autoclaving demonstrates glucocorticosteroid nanoparticles with an effective average particle size of less than about 2000 nm, and glucocorticosteroid chemical degradants accounting for less than 1% of the total glucocorticosteroid levels.

### 7. Methods of Making Aerosol Formulations

A nanoparticulate composition according to the invention for aerosol administration can be made by, for example, (1) nebulizing an aqueous dispersion of nanoparticulate composition according to the invention; (2) aerosolizing a dry powder of aggregates of a nanoparticulate composition according to the invention (the aerosolized composition may additionally contain a diluent); or (3) aerosolizing a suspension of a nanoparticulate aggregates of a composition according to the invention in a non-aqueous propellant. The aggregates of a nanoparticulate composition according to the invention, which may additionally contain a diluent, can be made in a non-pressurized or a pressurized non-aqueous system. Concentrated aerosol formulations may also be made by such methods.

### a. Spray-Dried Powder Aerosol Formulations

Spray drying is a process used to obtain a powder containing nanoparticulate drug particles following particle size reduction of a composition comprised of a nanoparticulate composition according to the invention in a liquid media. In general, spray-drying is used when the liquid media has a vapor pressure of less than about 1 atm at room temperature. A spray-dryer is a device which allows for liquid evaporation and powder collection. A liquid sample, either a solution or suspension, is fed into a spray nozzle. The nozzle generates droplets of the sample within a range of about 20 to about 100 μm (“micron”) in diameter which are then transported by a carrier gas into a drying chamber. The carrier gas temperature is typically between about 80 and about 200 degrees C. The droplets are subjected to rapid liquid evaporation, leaving behind dry particles which are collected in a special reservoir beneath a cyclone apparatus.

If the liquid sample consists of an aqueous dispersion of nanoparticles of a composition according to the invention, the collected product will consist of spherical aggregates of nanoparticles comprised of the composition according to the invention. If the liquid sample consists of an aqueous dispersion of nanoparticles in which an inert diluent material was dissolved (such as lactose or mannitol), the collected product will consist of diluent (e.g., lactose or mannitol) particles which contain an embedded nanoparticulate composition according to the invention. The final size of the collected product can be controlled and depends on the concentration of the nanoparticulate composition according to the invention and/or diluent in the liquid sample, as well as the droplet size produced by the spray-dryer nozzle. For deep lung delivery it is desirable for the collected product size to be less than about 2 microns in diameter, for delivery to the conducting airways it is desirable for the collected product size to be about 2 to about 6 microns in diameter, and for nasal delivery a collected product size of about 5 to about 100 μm is preferred. Compositions for ocular, otic, or topical delivery can vary in glucocorticosteroid particle size. Collected products may then be used in conventional DPIs for pulmonary or nasal delivery, dispersed in propellants for use in pMDIs, or the particles may be reconstituted in water for use in nebulizers.

In some instances, it may be desirable to add an inert carrier to the spray-dried material to improve the metering properties of the final product. This may especially be the case when the spray dried powder is very small (less than about 5 microns) or when the intended dose is extremely small, whereby dose metering becomes difficult. In general, such carrier particles (also known as bulking agents) are too large to be delivered to the lung and simply impact the mouth and throat and are swallowed. Such carriers typically consist of sugars such as lactose, mannitol, or trehalose. Other inert materials, including polysaccharides and cellulosics, may also be useful as carriers.

Spray-dried powders containing a nanoparticulate composition according to the invention may used in conventional DPIs, dispersed in propellants for use in pMDIs, or reconstituted in a liquid medium for use with nebulizers.

### b. Freeze-Dried Nanoparticulate Compositions

Sublimation, also known as freeze drying or lyophilization, can also be used to obtain a dry powder nanoparticulate composition. Sublimation can also increase the shelf stability of a composition according to the invention, particularly for biological products. Freeze-dried particles can also be reconstituted and used in nebulizers. Aggregates of freeze-dried nanoparticles of a composition according to the invention can be blended with either dry powder intermediates or used alone in DPIs and pMDIs for either nasal or pulmonary delivery.

Sublimation involves freezing the product and subjecting the sample to strong vacuum conditions. This allows for the formed ice to be transformed directly from a solid state to a vapor state. Such a process is highly efficient and, therefore, provides greater yields than spray-drying. The resultant freeze-dried product contains a composition according to the invention. The composition according to the invention is typically present in an aggregated state and can be used for inhalation alone (either pulmonary or nasal), in conjunction with diluent materials (lactose, mannitol, etc.), in DPIs or pMDIs, or reconstituted for use in a nebulizer.

### E. Methods of using the Nanoparticulate Glucocorticoid Compositions

The present invention provides a method of treating a mammal, including a human, requiring administration of a sterile dosage form of a glucocorticosteroid. The method comprises administering to a subject an effective amount of a sterile composition according to the invention.

The sterile compositions of the invention can be administered to a subject via any conventional means including, but not limited to, orally, rectally, ocularly, parenterally (e.g., intravenous, intramuscular, or subcutaneous), otic, intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

The sterile compositions of the invention, both aqueous and dry powder, are particularly useful in the treatment of respiratory-related illnesses such as asthma, emphysema, respiratory distress syndrome, chronic bronchitis, cystic fibrosis, chronic obstructive pulmonary disease, respiratory illness associated with acquired immune deficiency syndrome, and inflammatory and allergic conditions of the derma (skin), eye, and ear. The formulations and method result in improved surface area coverage of the application site (e.g., mouth, lung, nasal, eye, ear, etc.) by the administered composition according to the invention.

Administration by inhalation of glucocorticosteroids, compared with oral administration, reduces the risk of systemic side effects. The reduced risk of side effect arises from the mode of administration because glucocorticosteroids are highly active topically and only weakly active systemically, thereby minimizing effects on the pituitary-adrenal axis, the skin, and the eye. Side effects associated with inhalation therapy are primarily oropharyngeal candidiasis and dysphonia (due to atrophy of laryngeal muscles). Oral glucocorticosteroids cause atrophy of the dermis with thin skin, striae, and ecchymoses but inhaled glucocorticosteroids do not cause similar changes in the respiratory tract.

Other advantages of inhaled over oral administration include direct deposition of steroid in the airways which generally provides more predictable administration. The oral doses required for adequate control vary substantially, whereas inhaled glucocorticosteroids are usually effective within a narrower range. There are, however, a number of factors that influence the availability of inhaled glucocorticosteroids: extent of airway inflammation; degree of lung metabolism; amount of drug swallowed and metabolized in the GI tract; the patient's ability to coordinate the release and inspiration of the medication; type of glucocorticosteroid; and the delivery system.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, sodium chloride, Ringer's solution, lactated Ringer's solution, stabilizer solutions, tonicity enhancers (sucrose, dextrose, mannitol, etc.) polyols (propyleneglycol, polyethylene-glycol, glycerol, and the like), suitable mixtures thereof, vegetable oils (such as olive oil) and injectable organic esters such as ethyl oleate.

The nanoparticulate active agent compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the active agent, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

One of ordinary skill will appreciate that effective amounts of an active agent can be determined empirically and can be employed in pure form or, where such forms exist, in pharmaceutically acceptable salt, ester, or prodrug form. Actual dosage levels of an active agent in the nanoparticulate compositions of the invention may be varied to obtain an amount of active agent that is effective to obtain a desired therapeutic response for a particular composition and method of administration. The selected dosage level therefore, depends upon the desired therapeutic effect, the route of administration, the potency of the administered active agent, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

Both the foregoing general and detailed description are exemplary and explanatory and the following examples are intended to provide further explanation of the invention as claimed. Other objects, advantages, and novel features will be readily apparent to those skilled in the art from the following examples which are provided to more specifically set forth how to prepare and use the glucocorticosteroid formulations of the invention. It must be noted however, that they are for illustrative purposes only, and should not be deemed as limiting the spirit and scope of the invention as later recited in the claims.

### EXAMPLE 1

The purpose of this example was to evaluate the particle size of nanoparticulate dispersions of budesonide having polysorbate 80 as a nonionic surface stabilizer, both in the presence and absence of the amphiphilic lipid lecithin.

Budesonide has the following formula:

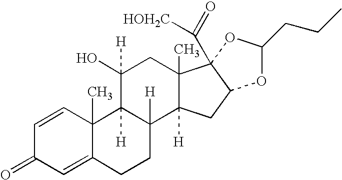

Budesonide is designated chemically as (RS)-11,16,17,21-Tetrahydroxy-pregna-1,4-diene-3,20-dione cyclic 16,17-acetal with butraldehyde. Budesonide is provided as the mixture of two epimers (22R and 22S). The empirical formula of budesonide is C₂₅H₃₄O₆ and its molecular weight is 430.5.

Budesonide is a white to off-white odorless powder that is practically insoluble in water and in heptane, sparingly soluble in ethanol, and freely soluble in chloroform.

An aqueous colloidal dispersion (NCD) containing 30% (w/w) budesonide and 1.5% (w/w) Polysorbate-80 was prepared by adding 10 g of Polysorbate-80 to 456.7 g Sterile Water for Injection (Abbott Labs) and 200 g of budesonide (Farmabios). The slurry was then combined with 593 g PolyMill™-500 (Dow Inc.) polymeric attrition media and charged into the 1215 mL chamber of a NanoMill®-1 milling system. The slurry was milled for 45 min. at 1000 rpm. Upon completion of the milling, the resulting milled budesonide/polysorbate-80 dispersion was harvested through a stainless steel screen. Particle size analysis of the budesonide/polysorbate-80 dispersion, using a Horiba LA-910 particle size analyzer (Irvine, Calif.), showed a mean particle size of 205 nm, with a D50 of 192 nm and a D90 of 291 nm. A portion of the 30% budesonide, 1.5% Polysorbate-80 dispersion was then further diluted with sterile water for injection to produce 20% (w/w), 10% (w/w), and 5% (w/w) budesonide containing 1% (w/w), 0.5% (w/w), and 0.25% (w/w) Polysorbate-80, respectively.

For Table I, separate portions of the 30% budesonide, 1.5% Polysorbate-80 dispersion were further compounded and diluted for preparation of:
(#1) 20% (w/w) budesonide, 0.33% (w/w) Lecithin NF (LIPOID), 1% (w/w) Polysorbate-80,
(#2) 10% (w/w) budesonide, 0.05% (w/w) Lecithin NF, 0.5% (w/w) Polysorbate-80 or
(#3) 5% (w/w) budesonide, 0.25% (w/w) Lecithin NF, 0.25% (w/w) Polysorbate 80.
   Lecithin NF is derived from soybean and is composed of a number of components, phosphatidylcholine, phosphatidylinositol, phosphatidylserine, and other lipid components. All of the resultant budesonide dispersions were placed in glass vials and sealed with aluminum crimped rubber stoppers, then steam heated in a Fedagari autoclave for 48.5 min. at 116° C. aluminum crimps.

Following the autoclave heat treatment, samples were examined for budesonide particle size in the Horiba LA-910 particle size analyzer with the results as shown in Table I.

TABLE I
Particle Size of Budesonide Dispersion Following Autoclave Heat
Treatment Effect of Polysorbate-80 alone or
Polysorbate-80 plus Lecithin-NF

| | Mean | D50 | D90 |
|---|---|---|---|
| Final Budesonide Formulation | (nm) | (nm) | (nm) |
| 20% Budesonide, 1% Polysorbate-80 | 668 | | 1492 |
| 10% Budesonide, 0.5% Polysorbate-80 | 776 | | 1854 |
| 5% Budesonide, 0.25% Polysorbate-80 | 879 | | 2213 |
| 20% Budesonide, 1% Polysorbate-80, 0.33% | 352 | | 504 |
| Lecithin NF | | | |
| 10% Budesonide, 0.5% Polysorbate-80, 0.5% | 346 | | 500 |
| Lecithin NF | | | |
| 5% Budesonide, 0.25% Polysorbate-80, 0.25% | 343 | | 493 |
| Lecithin NF | | | |

The results demonstrate that the presence of an amphiphilic lipid reduced particle size growth of the budesonide observed following autoclave heat treatment. The mean particle sizes of the budesonide formulations comprising an amphiphilic lipid was about half, or less, that of the budesonide formulations lacking an amphiphilic lipid. Moreover, even more dramatic results were obtained with measurement of the D90 particle size, demonstrating that the presence of an amphiphilic lipid effectively eliminated the growth of any large budesonide crystals following heat treatment.

### EXAMPLE 2

The purpose of this example was to determine the effect of different quantities of a nonionic surface stabilizer and a amphiphilic lipid on the particle size of a nanoparticulate budesonide dispersion following autoclave heat treatment.

Separate portions of the 30% budesonide, 1.5% Polysorbate-80 milled dispersion described in Example 1 were further diluted and compounded with the addition of varying levels of sterile water for injection (SWFI), Lecithin NF, and Polysorbate-80 to examine the effects of different percentages of Polysorbate-80 and Lecithin NF on budesonide particle size following autoclave heat treatment. The effects of different autoclave exposure temperatures is also illustrated in Table II (“API” is active pharmaceutical ingredient, or budesonide). All percentages in Table II are by weight.

TABLE II
Particle Size of Budesonide Dispersion Following Autoclave Heat
Treatment Effect of different percentages of Polysorbate-80 and Lecithin NF

| | | | | 15 min @ 121° C. | | | 48.5 min @ 116° C. | | |
|---|---|---|---|---|---|---|---|---|---|
| Final NCD Formulations | | | | Mean | D50 | D90 | Mean | D50 | D90 |
| Code | API | Polysorbate-80 | Lecithin | (nm) | (nm) | (nm) | (nm) | (nm) | (nm) |
| A | 20% | 1.00% | 0.20% | 345 | | 494 | 347 | | 498 |
| B | 20% | 1.00% | 0.10% | 346 | | 497 | 349 | | 502 |
| C | 20% | 1.00% | 0.05% | 356 | | 513 | 361 | | 529 |
| D | 20% | 3.00% | 0.20% | 52806 | | 163105 | 1159 | | 2815 |
| E | 20% | 3.00% | 0.10% | 47935 | | 155014 | 1103 | | 2593 |
| F | 20% | 3.00% | 0.05% | 3318 | | 3206 | 1065 | | 2440 |
| G | 10% | 0.50% | 0.50% | 343 | | 490 | 346 | | 496 |
| H | 10% | 0.50% | 0.10% | 347 | | 495 | 348 | | 498 |
| I | 10% | 0.50% | 0.50% | 345 | | 494 | 347 | | 498 |
| J | 10% | 1.50% | 0.50% | 350 | | 502 | 352 | | 506 |
| K | 10% | 1.50% | 0.10% | 350 | | 501 | 352 | | 504 |
| L | 10% | 1.50% | 0.05% | 351 | | 505 | 353 | | 507 |
| M | 10% | 3.50% | 0.50% | 396 | | 610 | 1510 | | 3654 |
| N | 10% | 3.50% | 0.10% | 2678 | | 6362 | 1653 | | 4147 |
| O | 10% | 3.50% | 0.05% | 1946 | | 4453 | 1731 | | 4062 |

The data show that higher percentages of Polysorbate-80 results in larger particle size growth during exposure to the autoclave heat treatment, as compared to lower percentages of Polysorbate-80. Higher percentages of Lecithin NF appear beneficial in producing smaller post-autoclave particle sizes.

### EXAMPLE 3

The purpose of this example was to determine the effect of phosphatide type on budesonide particle size following autoclave heat treatment.

An aqueous dispersion of 30% (w/w) budesonide and 1.5% (w/w) Polysorbate-80 was prepared by adding 12 g of Polysorbate-80 to 548 g Sterile Water for Injection (Abbott Labs) and 240 g of budesonide (Farmabios). The slurry was then combined with 474.3 g polyMill™-500 (Dow Inc) polymeric attrition media and charged into the 1215 mL chamber of a NanoMill®-1 milling system. The slurry was milled for 95 min. at 1200 rpm. Upon completion of the milling, the resulting nanoparticulate budesonide/polysorbate 80 dispersion was harvested through a stainless steel screen. Particle size analysis of the budesonide/polysorbate-80 dispersion, using a Horiba LA-910 particle size analyzer (Irvine, Calif.), showed a mean particle size of 197 nm, with a D50 of 185 nm and a D90 of 277 nm.

The resulting budesonide/polysorbate-80 dispersion was then diluted with Sterile Water for Injection and further compounded with disodium EDTA and one of a number of different phosphatides. Next, 10 g samples were placed in 20 cc glass vials and sealed with aluminum crimped rubber stoppers and steam heated in a Fedagari autoclave for 15 min. at 121° C. The various phosphatides examined in the formulation work represented Lecithin NF and examples purchased from the company, Lipoid, which included partially purified Lecithin (LIPOID S45), partially purified Hydrogenated Lecithin (LIPOID S75-3), purified Lecithin (LIPOID S100-3), Distearyl Phosphatidylethanolamine (PE 18:0/18:0), Distearyl Phosphatidylglycerol (PG 18:0/18:0) and Dipalmityl Phosphatidic Acid (PA 16:0/16:0).

Following the steam heat autoclave cycle, particle sizing was performed using the Horiba LA-910 with the results shown in Table III.

TABLE III
Particle Size of Budesonide Dispersion Following Autoclave Heat
Treatment: Effect of Phosphatide Type

| | | | | | | 15 min. @ 121° C. | | |
|---|---|---|---|---|---|---|---|---|
| | Formulation | | | | | Mean | D50 | D90 |
| Code | API | Polysorbate-80 | Lecithin | Type | EDTA | (nm) | (nm) | (nm) |
| A | 10% | 0.50% | 0.50% | Lecithin NF | 0.0010% | 350 | | 505 |
| C | 10% | 0.50% | 0.50% | Lipoid S 45 | 0.0010% | 350 | | 506 |
| D | 10% | 0.50% | 0.50% | Lipoid S 75-3 | 0.0010% | 353 | | 514 |
| E | 10% | 0.50% | 0.50% | Lipoid PG | 0.0010% | 384 | | 598 |
| | | | | 18:0/18:0 | | | | |
| G | 10% | 0.50% | 0.50% | Lipoid PA | 0.0010% | 343 | | 491 |
| | | | | 16:0/16:0 | | | | |
| B | 10% | 0.50% | 0.50% | Lipoid S 100-3 | 0.0010% | 18341 | | 52381 |
| F | 10% | 0.50% | 0.50% | Lipoid PE | 0.0010% | 16168 | | 56679 |
| | | | | 16:0/16:0 | | | | |

The results indicate that only impure mixtures of phosphatides (i.e., Lecithin NF, Lipoid S 45, or Lipoid S 75-3) and phosphatides which are negatively charged in these aqueous solutions (i.e., Lipoid PG 18:0/18:0 and Lipoid PA 16:0/16:0) are effective in maintaining small particle size and preventing particle size growth following exposure to the high temperatures during the autoclave cycle. In contrast, those phosphatides which are not negatively charged in aqueous solutions such as phosphatidylcholine (Lipoid S 100-3) or Lipoid PE 16:0/16:0 in combination with Polysorbate-80 lead to marked particle size growth following exposure to the autoclave heat treatment.

### EXAMPLE 4

The purpose of this example was to determine the resistance of a nanoparticulate budesonide dispersion to heat-induced chemical degradation of the budesonide and to determine if EDTA can provide additional protection against such degradation.

The NCD described in Example 3 was further compounded with Lecithin NF with and without EDTA to investigate the chemical stability of the budesonide dispersion following heat autoclave treatment. Fifty gram samples were autoclaved at 121° C. for 15, 25, and 35 min. with both the resulting particle size and level of total budesonide-related degradants determined. Table IV summarizes the total level of budesonide degradants as examined by HPLC for the three time periods of autoclave heat treatment.

TABLE IV
Resistance of Budesonide Dispersion to heat induced chemical
degradation: Additional Protection in the Presence of EDTA

| Formulation | | | | |
|---|---|---|---|---|
| 10% budesonide, 0.5% | No | 15 min @ 121° C. | 25 min @ 121° C. | 35 min @ 121° C. |
| Polysorbate-80, 0.5% | Autoclave | % Total | % Total | % Total |
| Lecithin NF | Treatment | Degradants | Degradants | Degradants |
| Autoclaved, EDTA absent | | 0.17% | 0.17% | 0.13% |
| Autoclaved, 0.0020% EDTA | | 0.12% | 0.12% | 0.12% |
| present | | | | |
| Not Autoclaved, EDTA | 0.12% | | | |
| absent | | | | |

The results demonstrate the resistance of each formulation with or without EDTA to chemical degradation of budesonide. However, the presence of EDTA offers a slight advantage in that a reduced level of total budesonide degradants was observed. A non-sterilized control had a total degradants level of 0.12%.

### EXAMPLE 5

The purpose of this example was to determine if dilution and further compounding of a glucocorticosteroid dispersion to concentration levels suitable for therapeutic use as an inhalation product has an effect on the particle size of the glucocorticosteroid.

An aqueous nanoparticulate budesonide dispersion (NCD) comprising 30% (w/w) budesonide and 1.5% (w/w) Polysorbate-80 was prepared by adding 12 g of Polysorbate-80 to 548 g Sterile Water for Injection (Abbott Labs) and 240 g of budesonide (Farmabios). The slurry was then combined with 474.3 g PolyMill™-500 (Dow Inc) polymeric attrition media and charged into the 1215 mL chamber of a NanoMill®-1 milling system. The slurry was milled for 95 min. at 1200 rpm. Upon completion of the milling, the resulting NCD was harvested through a stainless steel screen. Particle size analysis of the budesonide/polysorbate-80 dispersion, using a Horiba LA-910 particle size analyzer (Irvine, Calif.), showed a mean particle size of 197 nm, with a D50 of 185 nm and a D90 of 277 nm.

The resulting NCD was then diluted with Sterile Water for Injection, Lecithin NF, and disodium EDTA to prepare a formulation containing 10% (w/w) budesonide, 0.5% (w/w) Polysorbate-80, 0.5% (w/w) Lecithin NF, and 0.002% (w/w) EDTA. Ten gram aliquots of the formulation were placed in 20 cc glass vials and sealed with aluminum crimped rubber stoppers and steam heated in a Fedagari autoclave for 15 min. at 121° C. Following the autoclave heat treatment, each of the 10% (w/w) budesonide dispersions was then diluted with water, citric acid, sodium citrate, and additional Polysorbate-80 and disodium EDTA to produce dispersions containing either 0.1% budesonide or 0.0125% budesonide and varying levels of Polysorbate-80 and Lecithin NF.

The diluted and compounded samples were stored at room temperature for 7 days and then measured for particle size using the Horiba LA-910 particle size analyzer. The results are shown in Table V below.

TABLE V
Dilution and compounding of Budesonide NCD to Levels for Therapeutic
Usage as Inhalation Product: Retention of Small Dispersion Particel Size

| | Mean | D50 | D90 |
|---|---|---|---|
| Formulation | (nm) | (nm) | (nm) |
| 0.0125% API, 0.000625% Polysorbate-80, | 357 | | 508 |
| 0.000625% Lecithin NF, 0.02% Citric Acid, | | | |
| 0.03% Sodium Citrate, and 0.002% EDTA | | | |
| 0.0125% API, 0.002500% Polysorbate-80, | 356 | | 508 |
| 0.000625% Lecithin NF, 0.02% Citric Acid, | | | |
| 0.03% Sodium Citrate, and 0.002% EDTA | | | |
| 0.1% API, 0.005% Polysorbate-80, 0.005% | 356 | | 507 |
| Lecithin NF, 0.02% Citric Acid, 0.03% | | | |
| Sodium Citrate, and 0.002% EDTA | | | |
| 0.1% API, 0.020% Polysorbate-80, 0.005% | 353 | | 504 |
| Lecithin NF, 0.02% Citric Acid, 0.03% | | | |
| Sodium Citrate, and 0.002% EDTA | | | |

The results demonstrate that the nanoparticulate budesonide dispersion can be diluted and compounded to levels anticipated for usage as a therapeutic inhalation product without marked changes in the particle size of the dispersion

### EXAMPLE 6

The purpose of this example was to evaluate the sterility of a nanoparticulate budesonide dispersion following autoclave heat treatment.

Selected NCD preparations having been exposed to autoclave heat treatment cycles in either a Fedagari Model FOB2-3 or Getinge GEV-66 13 for varying time periods at 121° C. were evaluated for sterility using 6454 USP/EP Sterility by Direct Transfer with Transfer. The results of the sterility testing are tabulated in Table VI and meet the requirements as outlined in the current USP <71> sterility test and current EP w.6.1 sterility. There was no evidence of microbial growth upon completion of the incubation periods. The composition of the NCD autoclaved formulations were:
(1) R&D formulation #1 (in stainless steel bottles): 5% (w/w) budesonide, 0.25% (w/w) Polysorbate-80, 0.25% (w/w) LIPOID S75-3, 0.001% (w/w) EDTA, 94.5% (w/w) Water.
(2) R&D formulation #2 (in aluminum crimped stoppered glass vials): 10% (w/w) budesonide, 0.5% (w/w) Polysorbate-80, ?? % (w/w) Lecithin NF, ?? % (w/w) EDTA
(3) R&D formulation #3 (in aluminum crimped stoppered glass vials): 10% (w/w) budesonide, 0.5% (w/w) Polysorbate-80, 0.5% (w/w) LIPOID S75-3, 0.001% (w/w) EDTA, 89% (w/w) Water.
(4) R&D formulation #4 (in stainless steel bottles): 5% (w/w) budesonide, 0.25% (w/w) Polysorbate-80, 0.25% (w/w) Lipoid S75-3, 0.001% EDTA, 94.5% (w/w) Water.

(5) GMP formulation #5: 5% (w/w) budesonide, 0.25% (w/w) Polysorbate-80, 0.25% (w/w) Lipoid S75-3, 0.001% (w/w) EDTA, 94.5% (w/w) Sterile Water for Injection

TABLE VI
Sterility of Budesonide Dispersions Following Heat Autoclave

| | 10 min @ 121° C. | 15 min @ 121° C. | 20 min @ 121° C. | 25 min @ 121° C. | 35 min @ 121° C. |
|---|---|---|---|---|---|
| | Sterility | Sterility | Sterility | Sterility | Sterility |
| Formulation | Result | Result | Result | Result | Result |
| Formulation #1 | | No visible | No visible | | |
| | | growth | growth | | |
| Formulation #2 | | No visible | | No visible | No visible |
| | | growth | | growth | growth |
| Formulation #3 | | | No visible | | |
| | | | growth | | |
| Formulation #4 | No visible | | | | |
| | growth | | | | |
| Formulation #5 | | No visible | | | |
| | | growth | | | |

### EXAMPLE 7

The purpose of this example was to evaluate the particle size of nanop articulate dispersions of the beclomethasone dipropionate having Polysorbate-80 as a non-ionic surface stabilizer both in the presence and absence of the amphiphilic lipid, LIPOID 45 or LIPOID S75-3.

Beclomethasone dipropionate has the following structural formula:

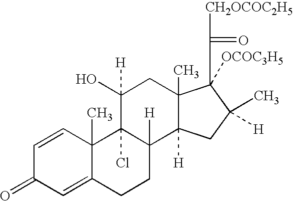

It is a white powder with a molecular weight of 521.25 and is very slightly soluble in water.

An aqueous nanoparticulate dispersion (NCD) comprising 10% (w/w) beclomethasone and 0.5% Polysorbate-80 (w/w) was prepared by milling in a DynoMill® System utilizing PolyMill™-500 (Dow Inc) polymeric attrition media, with milling for 40 minutes. Particle size analysis of the beclomethasone/polysorbate-80 dispersion, using a Horiba LA-910 particle size analyzer (Irvine, Calif.), indicated agglomeration, with a mean particle size of 30503 nm. Additional Polysorbate-80 was spiked into the formulation to yield 10% (w/w) beclomethasone and 1.0% Polysorbate-80 (w/w). Milling was resumed for 5 minutes then re-analyzed for particle size, which indicated a mean particle size of 272 nm, with a D50 of 254 nm and a D90 of 386 nm.

The resulting nanoparticulate beclomethasone/polysorbate-80 dispersion was then diluted to prepare three separate formulations, namely:

(1) 5% (w/w) beclomethasone, 0.5% (w/w) Polysorbatee-80, and 0.5% (w/w) LIPOID S45;

(2) 5% (w/w) beclomethasone, 0.5% (w/w) Polysorbate-80, and 0.25% (w/w) LIPOID S75-3; and

(3) 5% (w/w) beclomethasone, 0.5% (w/w) Polysorbate-80, and 0.5% (w/w) LIPOID S75-3.

All of the resultant NCD samples were placed in glass vials and sealed with rubber stoppers and aluminum crimps, followed by autoclave heat treatment in a Fedagari autoclave for 10 min at 121.1° C. Following the autoclave heat treatment, samples were examined for particle size in the Horiba LA-910 particle size analyzer with the results as shown in Table VII.

TABLE VII
Particle Size of Beclomethasone Dispersion Following Autoclave
Heat Treatment Effect of Polysorbate-80 alone and Polysorbate-80
plus Lipoid S75-3

| | Mean | D50 | D90 |
|---|---|---|---|
| Formulation | (nm) | (nm) | (nm) |
| 10% Beclomethasone, 1% | 5336 | | 10260 |
| Polysorbate-80 | | | |
| 5% Beclomethasone, 0.5% | 2539 | | 5056 |
| Polysorbate-80, 0.5% LIPOID S45 | | | |
| 5% Beclomethasone, 0.5%, | 2432 | | 4736 |
| Polysorbate-80, 0.25% LIPOID S75-3 | | | |
| 5% Beclomethasone, 0.5% | 2404 | | 4670 |
| Polysorbate-80, 0.5% LIPOID S75-3 | | | |

### EXAMPLE 8

The purpose of this example was to determine the effect of the nonionic surface stabilizer tyloxapol alone as compared to tyloxapol in combination with an amphiphilic lipid on the particle size of beclomethasone following autoclaveheat treatment.

An aqueous nanoparticulate dispersion (NCD) of beclomethasone having 10% (w/w) beclomethasone and 1.0% (w/w) tyloxapol was prepared by milling in a DynoMill® System utilizing polyMill™-500 (Dow Inc) polymeric attrition media, with milling for 30 minutes. Particle size analysis of the beclomethasone/tyloxapol dispersion, using a Horiba LA-910 particle size analyzer (Irvine, Calif.), showed a mean particle size of 146 nm, with a D50 of 141 nm and a D90 of 201 nm.

The resulting NCD was then diluted to prepare four separate formulations, namely:
(1) 5% (w/w) beclomethasone, 0.5% (w/w) tyloxapol;
(2) 5% (w/w) beclomethasone, 0.5% (w/w) tyloxapol, and 0.5% (w/w) Lecithin NF;
(3) 5% (w/w) beclomethasone, 0.5% (w/w) tyloxapol, and 0.25% (w/w) Lecithin NF; and
(4) 5% (w/w) beclomethasone, 0.5% (w/w) tyloxapol, and 0.25% (w/w) LIPOID S75-3.

All of the samples were placed in crimp-top rubber-stoppered vials and steam sterilized for 10 minutes at 1211° C. The post-sterilization particle sizes are shown in Table VIII below.

TABLE VIII
Particle Size of Beclomethasone Dispersion Following Autoclave
Heat Treatment: Effect of Tyloxapol alone or Tyloxapol plus Phosphatide

| | Mean | D50 | D90 |
|---|---|---|---|
| Formulation | (nm) | (nm) | (nm) |
| 5% Beclomethasone, 0.5% Tyloxapol | 3251 | | 6757 |
| 5% Beclomethasone, 0.5% Tyloxapol, | 785 | | 1255 |
| 0.5% Lecithin NF | | | |
| 5% Beclomethasone, 0.5% Tyloxapol, | 795 | | 1274 |
| 0.25% Lecithin NF | | | |
| 5% Beclomethasone, 0.5% Tyloxapol, | 779 | | 1268 |
| 0.25% LIPOID S75-3 | | | |

### EXAMPLE 9

The purpose of this example was to determine the effect of a non-ionic surface stabilizer in combination with an amphiphilic lipid on the particle size of the glucocorticosteroid fluticasone propionate following autoclaveheat treatment.

Fluticasone propionate has the chemical name S-(fluoromethyl) 6a,9-difluoro-11b, 17-dihydroxy-16a-methyl-3-oxoandrosta-1,4-diene-17b-carbothioate, 17-propionate and the following chemical structure:

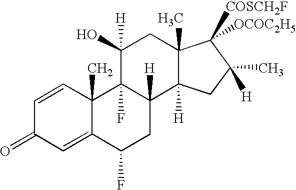

Fluticasone propionate is a white to off-white powder with a molecular weight of 500.6, and the empirical formula C₂₅H₃₁F₃O₅S. It is practically insoluble in water.

An aqueous nanoparticulate dispersion (NCD) of fluticasone having 10% (w/w) fluticasone and 0.5% (w/w) Polysorbate-80 (w/w) was prepared by milling in a DynoMill® System utilizing PolyMill™-500 (Dow Inc) polymeric attrition media for 25 minutes. Particle size analysis of the fluticasone/polysorbate-80 dispersion, using a Horiba LA-910 particle size analyzer (Irvine, Calif.), indicated agglomeration, with a mean particle size of 23145 nm.

Additional Polysorbate-80 was spiked into the formulation to yield 10% (w/w) fluticasone and 1.0% (w/w) Polysorbate-80 (w/w). Milling was continued for 5 minutes before re-analysis, which continued to display a large particle size (Dmean of 20675 nm).

Lecithin NF was spiked into the formulation to yield 10% (w/w) fluticasone, 1.0% (w/w) Polysorbate-80, and 0.5% (w/w) Lecithin NF. Milling was continued for 10 minutes. The final mean particle size was 171 nm, with a D50 of 164 nm and a D90 of 232 nm.

The resulting NCD was then diluted to 5% (w/w) fluticasone, 0.5% (w/w) Polysorbate-80, and 0.5% (w/w) Lecithin NF. Both samples were placed in aluminum crimp-top rubber-stoppered vials and steam heated in a Fedagari autoclave for 10 minutes at 121.1° C. The post-sterilization particle sizes are shown in Table IX below.

TABLE IX
Particle Size of Fluticasone Dispersion Following Autoclave Heat
Treatment: Effect of Polysorbate-80 plus Lecithin NF

| | Mean | D50 | D90 |
|---|---|---|---|
| Formulation | (nm) | (nm) | (nm) |
| 10% Fluticasone, 1.0% Polysorbate- | 306 | | 431 |
| 80, 0.5% Lecithin NF | | | |
| 5% Fluticasone, 0.5% Polysorbate- | 312 | | 439 |
| 80, 0.5% Lecithin NF | | | |

### EXAMPLE 10

The purpose of this example was to determine the effect of the nonionic surface stabilizer Lutrol F127 NF as compared to Lutrol F127 NF in combination with an amphiphilic lipid, Lecithin NF or LIPOID S75-3 on the particle size of budesonide following autoclave heat treatment.

An aqueous nanoparticulate dispersion (NCD) of budesonide having 10% (w/w) budesonide and 1.0% (w/w) Lutrol F127 NF was prepared by milling in a DynoMill® System utilizing polyMill™-500 (Dow Inc) polymeric attrition media for 40 minutes. Particle size analysis of the budesonide/Lutrol F127 NF dispersion, using a Horiba LA-910 particle size analyzer (Irvine, Calif.), showed a mean particle size of 221 nm, with a D50 of 202 nm and a D90 of 324 nm. The resulting NCD was then diluted to prepare three separate formulations, namely:
(1) 5% (w/w) budesonide, 0.5% (w/w) Lutrol F127 NF, and 0.5% (w/w) Lecithin NF;
(2) 5% (w/w) budesonide, 0.5% (w/w) Lutrol F127 NF, 0.25% (w/w) Lecithin NF; and
(3) 5% (w/w) budesonide, 0.5% (w/w) Lutrol F127 NF, 0.25% (w/w) LIPOID S75-3.

All of the samples were placed in aluminum crimp-top rubber-stoppered vials and steam heated in a Fedagari autoclave for 10 minutes at 121.1° C. The post-sterilization particle sizes are shown in Table IX below.

TABLE IX
Particle Size of Budesonide Dispersion Following Autoclave Heat
Treatment: Effect of Lutrol F127 NF and Lutrol F127 NF plus Lecithin NF

| | Mean | D50 | D90 |
|---|---|---|---|
| Formulation | (nm) | (nm) | (nm) |
| 10% Budesonide, 1% Lutrol F127 NF | 1141 | | 2589 |
| 5% Budesonide, 0.5% Lutrol F127 NF, | 838 | | 1748 |
| 0.5% Lecithin NF | | | |
| 5% Budesonide, 0.5% Lutrol F127 NF, | 863 | | 1788 |
| 0.25% Lecithin NF | | | |
| 5% Budesonide, 0.5% Lutrol F127 NF, | 936 | | 1967 |
| 0.25% LIPOID S75-3 | | | |

The results indicate that the presence of an amphiphilic lipid during the autoclave treatment significantly reduces the particle size of the budesonide dispersion.

The purpose of this example was to determine the effect of tyloxapol as compared to tyloxapol in combination with lecithin NF on the particle size of budesonide following autoclaveheat treatment.

An aqueous nanoparticulate dispersion (NCD) of budesonide having 10% (w/w) budesonide and 1.0% (w/w) tyloxapol was prepared by milling in a DynoMill® System utilizing PolyMill™-500 (Dow Inc) polymeric attrition media for 30 minutes. Particle size analysis of the budesonide/tyloxapol dispersion, using a Horiba LA-910 particle size analyzer (Irvine, Calif.), showed a mean particle size of 159 nm, with a D50 of 152 nm and a D90 of 221 nm. The resulting NCD was then diluted to prepare four separate formulations, namely:
(1) 5% (w/w) budesonide and 0.5% (w/w) tyloxapol;
(2) 5% (w/w) budesonide, 0.5% (w/w) tyloxapol, and 1.0% (w/w) Lecithin NF;
(3) 5% (w/w) budesonide, 0.5% (w/w) tyloxapol, and 0.5% (w/w) Lecithin NF; and
(4) 5% (w/w) budesonide, 0.5% (w/w) tyloxapol, and 0.25% (w/w) Lecithin NF.

All of the samples were placed in aluminum crimp-top rubber-stoppered vials and steam heated in a Fedagari autoclave for 10 minutes at 121.1 ° C. The post-autoclaved particle sizes are shown in Table X below.

TABLE X
Particle Size of Budesonide Dispersion Following Autoclave Heat
Treatment: Effect of Tyloxapol and Tyloxapol plus Lecithin NF

| | Mean | D50 | D90 |
|---|---|---|---|
| Formulation | (nm) | (nm) | (nm) |
| 5% Budesonide, 0.5% Tyloxapol | 4806 | | 5777 |
| 5% Budesonide, 0.5% Tyloxapol, 1.0% | 406 | | 697 |
| Lecithin NF | | | |
| 5% Budesonide, 0.5% Tyloxapol, 0.5% | 401 | | 689 |
| Lecithin NF | | | |
| 5% Budesonide, 0.5% Tyloxapol, | 410 | | 712 |
| 0.25% Lecithin NF | | | |

The results demonstrate that the presence of an amphiphilic lipid, in combination with a non-ionic surface stabilizer, dramatically reduces the heat sterilized glucocorticosteroid particle size.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A sterile composition comprising:
(a) particles of at least one glucocorticosteroid, wherein the particles have an effective average particle size of less than about 2000 nm;
(b) at least one nonionic surface stabilizer; and
(c) at least one amphiphilic lipid.

2. The composition of claim 1, wherein the composition is sterilized by moist heat sterilization.

3. The composition of claim 2, wherein the sterilizing temperature is from about 110° C. to about 135° C.

4. The composition of claim 1, wherein the glucocorticosteroid is selected from the group consisting of budesonide, triamcinolone acetonide, triamcinolone, mometasone, mometasone furoate, flunisolide, fluticasone propionate, fluticasone, beclomethasone dipropionate, dexamethasone, triamincinolone, beclomethasone, fluocinolone, fluocinonide, flunisolide hemihydrate, mometasone furoate monohydrate, clobetasol, and combinations thereof.

5. The composition of claim 1, wherein the nonionic surface stabilizer is selected from the group consisting of sorbitol esters, polyoxyethylene sorbitan esters, poloxamers, polysorbates, spans, sorbitan oleate esters, sorbitan palmitate esters, sorbitan stearate esters, polyoxyethylene sorbitan monolaurate, polyoxyethylene sorbitan monooleate, glyceryl monooleate, glyceryl mono-laurate, surfactants containing polyethylene oxide chains, polysorbate 80, polysorbate 60, poloxamer 407, Pluronic® F68, Pluronic®F108, Pluronic®F127, hydroxypropyl methylcellulose, hydroxypropylcellulose, polyvinylpyrrolidone, random copolymers of vinyl pyrrolidone and vinyl acetate, dextran, cholesterol, polyoxyethylene alkyl ethers, macrogol ethers, cetomacrogol 1000, polyoxyethylene castor oil derivatives, polyethylene glycols, Carbowax 3550®, Carbowax 934®, polyoxyethylene stearates, methylcellulose, hydroxyethylcellulose, noncrystalline cellulose, polyvinyl alcohol, tyloxapol, poloxamers, p-isononylphenoxypoly-(glycidol), C₁₈H₃₇CH₂C(O)N(CH₃)—CH₂(CHOH)₄(CH₂0H)₂; decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, and mixtures thereof.

6. The composition of claim 5, wherein the nonionic surface stabilizer is selected from the group consisting of poloxamer 407, polysorbate 80, polysorbate 60, tyloxapol, and block copolymers of ethylene oxide and propylene oxide.

7. The composition of claim 6, wherein the nonionic surface stabilizer is selected from the group consisting of Pluronic® F68, Pluronic® F108, and Pluronic® F127.

8. The composition of claim 1, wherein the amphiphilic lipid is a phospholipid containing at least one negatively charged phospholipids.

9. The composition of claim 8, wherein the phospholipid is selected from the group consisting of anionic phosphatides, lecithin NF, synthetic lecithin NF, synthetic phospholipids, partially purified hydrogenated lecithin, hydrogenated lecithin, partially purified lecithin, soy lecithin phosphatides comprising anionic phophatides, egg lecithin phosphatides comprising anionic phophatides, hydrogenated soy lecithins comprising anionic phosphatides, hydrogenated egg lecithins comprising anionic phosphatides, lecithins comprising anionic phosphatides, synthetic phosphatidyl glycerol, synthetic phosphatidic acid, synthetic phosphatidyl inositol, synthetic phosphatidyl serine, phosphatidyl inositol, phosphatidyl serine, phosphatidic acid, phosphatidyl glycerol, lysophosphatidyl inositol, lysophosphatidyl serine, lysophosphatidic acid, lysophosphatidyl glycerol, distearyl phosphatidyl glycerol, distearyl phosphatidyl inositol, distearyl phosphatidyl serine, distearyl phosphatidic acid, distearyl lysophosphatidyl glycerol, distearyl lysophosphatidyl inositol, distearyl lysophosphatidyl serine, distearyl lysophosphatidic acid, dipalmityl phosphatidyl inositol, dipalmityl phosphatidyl serine, dipalmityl phosphatidic acid, dipalmityl phosphatidyl glycerol, dipalmityl lysophosphatidyl inositol, dipalmityl lysophosphatidyl serine, dipalmityl lysophosphatidic acid, dipalmityl lysophosphatidyl glycerol, and mixtures thereof.

10. The composition of claim 9, wherein the phospholipid is lecithin, and the lecithin comprises less than 90% phosphatidylcholine.

11. The composition of claim 10, wherein the lecithin is comprised substantially of hydrogenated phosphatidylcholine and the remaining composition composed of mainly hydrogenated anionic phosphatides.

12. The composition of claim 1, wherein the chemical purity of the glucocorticosteroid is greater than 99%.

13. The composition of claim 1, wherein the chemical purity of the glucocorticosteroid is greater than 99.5%.

14. The composition of claim 1, wherein the amount of the glucocorticosteroid, in concentrated form or upon dilution in a pharmaceutically acceptable vehicle, ranges from about 0.01% to about 20% by weight.

15. The composition of claim 1, further comprising sodium salt of ethylenediaminetetraacetic acid, calcium salt of ethylenediaminetetraacetic acid, or a combination thereof.

16. The composition of claim 15, wherein the amount of sodium salt and/or calcium salt of ethylenediaminetetraacetic acid ranges from about 0.0001% to about 5%, from about 0.001 to about 1%, and from about 0.01% to about 0.1%

17. The composition of claim 1, wherein the concentration of the nonionic surface stabilizer is selected from the group consisting of from about 0.01% to about 90%, from about 0.1% to about 50%, and from about 1% to about 10%, by weight, based on the total combined dry weight of the glucocorticosteroid and the surface stabilizer.

18. The composition of claim 1, wherein the effective average particle size of the glucocorticosteroid particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

19. The composition of claim 18, wherein at least about 70%, at least 80%, at least about 90%, at least about 95%, or at least about 99% of the glucocorticosteroid particles, by weight, have a particle size of less than the effective average.

20. The composition of claim 1, further comprising one or more pharmaceutically acceptable excipients.

21. The composition of claim 1 in a dosage form:
(a) formulated for inhalation, injectable, otic, oral, rectal, pulmonary, opthalmic, colonic, parenteral, intracisternal, intravaginal, intraperitoneal, local, buccal, nasal, or topical administration;
(b) formulated into a powder, lyophilized powder, spray dried powder, spray granulated powder, solid lozenge, capsule, tablet, pill, granule, liquid dispersion, gel, aerosol, ointment, or cream;
(c) formulated into a dosage form selected from the group consisting of controlled release formulation, solid dose fast melt formulation, controlled release formulations, fast melt formulations, lyophilized formulations, delayed release formulations, extended release formulations, pulsatile release formulations, and mixed immediate release and controlled release formulations; or (d) any combination of (a), (b), and (c).

22. The composition of claim 1, formulated into a nasal spray.

23. The composition of claim 1, formulated into a pulmonary aerosol.

24. The composition of claim 1, formulated into an aqueous aerosol and comprising from about 0.025 mg/mL up to about 600 mg/mL of the glucocorticosteroid.

25. The aerosol composition of claim 24, wherein glucocorticosteroid concentration is selected from the group consisting of about 10 mg/mL or more, about 100 mg/mL or more, about 200 mg/mL or more, about 400 mg/mL or more, and about 600 mg/mL.

26. The composition of claim 1, formulated into an aqueous aerosol, wherein the droplets of the aerosol have a mass median aerodynamic diameter selected from the group consisting of less than or equal to about 100 microns; from about 0.1 to about 10 microns; from about 2 to about 6 microns; less than about 2 microns; from about 5 to about 100 microns; and from about 30 to about 60 microns.

27. The composition of claim 1 formulated into an aerosol and further comprising one or more solvents and/or propellants dissolved in a non-aqueous solution for co-administration from a multi-dose inhaler.

28. The composition of claim 1, further comprising at least one non-glucocorticosteroid active agent.

29. The composition of claim 28, wherein the at least one non-glucocorticosteroid active agent is useful in treating asthma, allergic conjunctivitis, seasonal allergic rhinitis, or other inflammatory or allergic condition for which glucocorticosteroids are conventionally used.

30. The composition of claim 28, wherein the non-glucocorticosteroid active agent is selected from the group consisting of long-acting beta-agonists, leukotriene modifiers, theophylline, nedocromil, cromolyn, short-acting beta-agonists, ipratropium bromide, prednisone, prednisolone, methylprednisolone, salmeterol, formoterol, monoleukast, zafirlukast, zileuton, albuterol, levalbuterol, bitolterol, pirbuterol, and terbutaline.

31. The composition of claim 1, formulated into an aqueous aerosol wherein:
(a) essentially each droplet of the aqueous aerosol comprises at least one nanoparticulate glucocorticosteroid particle;
(b) the droplets of the aerosol have a mass median aerodynamic diameter (MMAD) less than or equal to about 100 microns;
(c) the glucocorticosteroid is selected from the group consisting of fluticasone, budesonide, triamcinolone acetonide, triamcinolone, mometasone, mometasone furoate, fluticasone propionate, beclomethasone dipropionate, dexamethasone, triamincinolone, beclomethasone, fluocinolone, fluocinonide, flunisolide hemihydrate, flunisolide, mometasone furoate monohydrate, clobetasol, and combinations thereof;
(d) the glucocorticosteroid is present in a concentration of from about 0.05 mg/mL up to about 600 mg/mL
(e) the nonionic stabilizer is a polyoxyethylene sorbitan fatty acid ester; and
(f) the amphiphilic lipid is a phospholipid.

32. A method of making a sterile composition comprising:
(a) particles of at least one glucocorticosteroid, wherein the particles have an effective average particle size of less than about 2000 nm;
(b) at least one nonionic surface stabilizer; and
(c) at least one amphiphilic lipid,
wherein the method comprises:
(i) contacting particles of a glucocorticosteroid with at least one nonionic surface stabilizer for a time and under conditions to reduce the effective average particle size of the particles to less than about 2000 nm;
(ii) adding at least one amphiphilic lipid to the glucocorticosteroid composition, either before, during, or after particle size reduction; and
(iii) steam heating the composition to a temperature of from about 115° C. to about 135° C.

33. A method of treating a subject in need comprising administering to the subject a therapeutically effective amount of a sterile composition comprising:
(a) particles of at least one glucocorticosteroid, wherein the particles have an effective average particle size of less than about 2000 nm;
(b) at least one nonionic surface stabilizer; and
(c) at least one amphiphilic lipid.

34. The method of claim 33, wherein the composition comprises at least one pharmaceutical excipient or carrier.

35. The method of claim 33, wherein said treatment is for an inflammatory disease.

36. The method of claim 33, wherein the treatment is for asthma, cystic fibrosis, chronic obstructive pulmonary disease, emphysema, respiratory distress syndrome, chronic bronchitis, respiratory illness associated with acquired immune deficiency syndrome, and inflammatory conditions of the eye, inflammatory conditions of the skin, inflammatory conditions of the ear, allergic conditions of the eye, allergic conditions of the skin, allergic conjunctivitis, and seasonal allergic rhinitis.

37. The method of claim 33, wherein the composition is administered via a nasal or pulmonary aerosol.

38. The method of claim 37 wherein the patient delivery time for the aerosol administration is from about 15 seconds up to about 15 minutes.

39. A sterile composition comprising:
(a) particles of at least one glucocorticosteroid, wherein the particles have an effective average particle size of less than about 2000 nm;
(b) at least one nonionic surface stabilizer;
(c) at least one amphiphilic lipid; and
(d) ethylenediaminetetraacetic acid, a sodium salt of ethylenediaminetetraacetic acid, a calcium salt of ethylenediaminetetraacetic acid, or a combination thereof,
wherein the ethylenediaminetetraacetic acid or salt thereof is present in a amount sufficient to prevent or reduce heat-induced chemical degradation of one or more components in the composition.

40. The composition of claim 1, wherein the ethylenediaminetetraacetic acid or salt thereof is present in an amount sufficient to prevent or reduce heat-induced catalytic oxidation of the glucocorticosteroid in the composition.

41. The composition of claim 1, wherein the amount of ethylenediaminetetraacetic acid or salt thereof sufficient to prevent or reduce heat-induced chemical degradation of one or more components in the composition is at least 0.0001% w/w.
